(19) United States
(12) Patent Application Publication
(10) Pub. No.: US 2020/0246312 A1
(43) Pub. Date: Aug. 6, 2020

(54) ORAL BENDAMUSTINE FORMULATIONS

(71) Applicant: TUBE Pharmaceuticals GmbH, Wien (AT)

(72) Inventor: Wolfgang RICHTER, Wien (AT)

(21) Appl. No.: 16/753,690

(22) PCT Filed: Oct. 5, 2018
(86) PCT No.: PCT/EP2018/077214

§ 371 Date: Apr. 3, 2020

(30) Foreign Application Priority Data
Oct. 5, 2017 (EP) 17194987.8

(51) Int. Cl. A61K 31/4184; A61K 47/40; C07K 16/28; A61K 9/00

(52) U.S. Cl. CPC A61K 31/4184; A61K 47/40; A61K 45/06; A61K 9/0053; C07K 16/2887

(57) ABSTRACT

The present invention relates to an oral pharmaceutical composition comprising bendamustine in combination with a modified cyclodextrin, such as, e.g., methyl-ß-cyclodextrin or hydroxypropyl-ß-cyclodextrin. It has surprisingly been found in the context of the invention that such compositions exhibit a greatly improved oral bioavailability, which renders them particularly advantageous for oral therapeutic application, e.g., in the treatment of cancer.

The present invention relates to an oral pharmaceutical composition comprising bendamustine in combination with a modified cyclodextrin, such as, e.g., methyl-β-cyclodextrin or hydroxypropyl-β-cyclodextrin. It has surprisingly been found in the context of the invention that such compositions exhibit a greatly improved oral bioavailability, which renders them particularly advantageous for oral therapeutic application, e.g., in the treatment of cancer.

Bendamustine hydrochloride [i.e., 4-(5-(bis-(2-chloro-ethyl)-am ino)-1-methyl-1H-benzimidazol-2-yl)-butyric acid hydrochloride] was synthesized for the first time in 1963 by Ozegowski et al. and developed in the 1960's by Jenapharm in the former German Democratic Republic (GDR) as an anticancer drug (Ozegowski W et al., J Prakt Chem, 1963, 20, 178-186; Ozegowski W et al., Zentralbl Pharm, 1971, 110, 1013-1019). Bendamustine combines the alkylating activity of the nitrogen mustard group and the antimetabolite properties of the benzimidazole scaffold. It has a di-(chloroethyl)-amine group which can potentially give crosslinking alkylation of DNA strands.

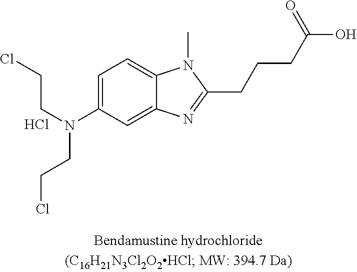 [SMILES: Cn1c(CCCC(=O)O)nc2cc(N(CCCl)CCCl)ccc21 ; Cl]

The nitrogen mustard group of bendamustine is prone to chemical hydrolysis, in particular at neutral or basic pH values thus resulting in the formation of the mostly inactive mono- and 5-(bis-(2-hydroxyethyl)-amino)-substituted bendamustine derivatives (see Scheme 1). The hydrolytic decomposition is prevented or reduced at acidic pH because the protonation of the nitrogen atom leads to decreased bendamustine nucleophilicity and, consequently, the tendency to form an aziridinium ion is considerably lowered. The hydrolysis rate is also retarded in the presence of high concentrations of chloride (Maas B et al., Pharmazie, 1994, 49(10), 775-777).

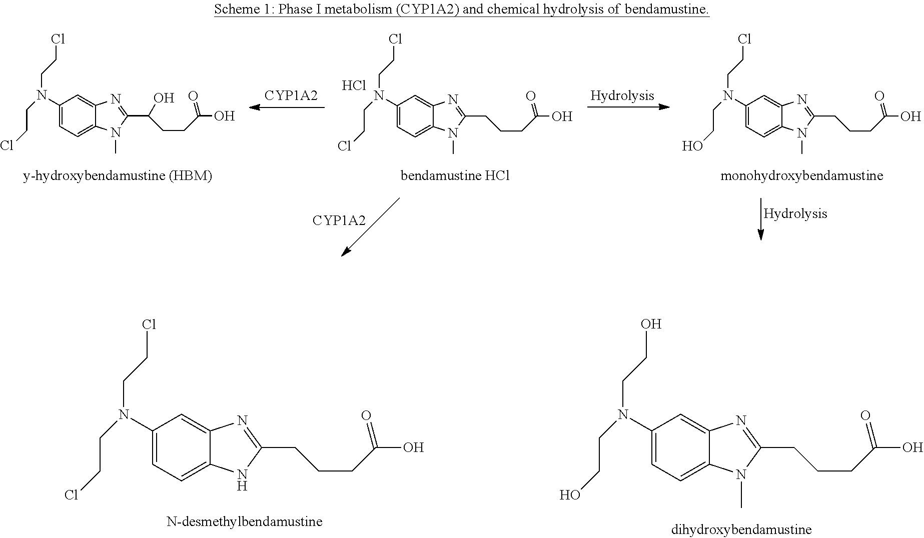 [SMILES: Cn1c(C(O)CCC(=O)O)nc2cc(N(CCCl)CCCl)ccc21 ; Cl ; Cn1c(CCCC(=O)O)nc2cc(N(CCCl)CCCl)ccc21 ; Cn1c(CCCC(=O)O)nc2cc(N(CCO)CCCl)ccc21 ; O=C(O)CCCc1nc2cc(N(CCCl)CCCl)ccc2[nH]1 ; Cn1c(CCCC(=O)O)nc2cc(N(CCO)CCO)ccc21]

In addition to the chemical hydrolysis of the nitrogen mustard group which starts already during administration of the drug to the patient the formation of phase I and phase II metabolites of bendamustine occurs in plasma and are described in literature (Darwish M et al., Cancer Chemother Pharmacol, 2015, 75, 1143-1154). Especially CYP1A2 was assumed to be relevant for the formation of the two known active phase I metabolites, N-desmethyl-bendamustine and γ-hydroxybendamustine (M3 metabolite) (see Scheme 1) (Teichert J et al., Cancer Chemother Pharmacol, 2007, 59(6), 759-770). With regard to phase II metabolites, biliary excretion of N-acetyl-L-cysteine conjugates was previously described as a mechanism of bendamustine elimination (Teichert J et al., Drug Metab Dispos, 2005, 33(7), 984-992; Teichert J et al., Drug Metab Dispos, 2009, 37(2), 292-301).

First clinical trials with bendamustine were performed in 1965 for the treatment of several indications in oncology, especially hematological malignancies, breast cancer, lung cancer and ovarian cancer. Jenapharm registered a powder for injection formulation in the former East Germany as Cytostasan in 1971. After the German reunification, the product has been marketed in its current formulation since 1991, mostly under the trade name Ribomustin™ and thus registered in Europe. Bendamustine hydrochloride powder for injection has been available in the USA since 2008 under the trade name Treanda™, registered by Teva (Werner W et al., Onkologie, 2013, 36(Suppl 1), 2-10). Published information indicates that a different, “fast and convenient” formulation was developed in 2013 by Eagle Pharmaceuticals, Inc. The new intravenous formulation is now available as 100 mg/4 mL non-aqueous solution formulated with propylene glycol, polyethylene glycol 400 and monothioglycerol.

Bendamustine is approved for the treatment of chronic lymphocytic leukemia (CLL), indolent non-Hodgkin lymphoma (indolent NHL) and multiple myeloma (MM) in Germany (Levact™) and for CLL and indolent NHL in the US (Treanda™). Most treatment regimens apply bendamustine in combination with other cytostatic drugs, often with rituximab. Clinical research on bendamustine has been intensified in the last years. However, most trials with bendamustine as a single agent, for instance, for the treatment of bile duct cancer, soft tissue sarcoma, germ cell cancer, small cell lung cancer, pretreated metastatic or advanced breast cancer revealed on the one hand good tolerability, but on the other hand limited benefit. Studies on bendamustine in combination therapy, for example with methotrexate and 5-fluorouracil for the treatment of metastatic breast cancer or bendamustine with carboplatin for the treatment of small cell lung cancer, reported efficacies which were comparable to respective standard treatment regimens. Due to mild side effects and reduced cross resistances with other alkylating drugs, bendamustine was claimed to be an interesting drug for the treatment of patients in poor clinical condition or as second line therapy.

So far, only intravenous formulations are available for bendamustine and no oral formulations are on the market although bendamustine itself has an oral bioavailability of about 56% (Preiss R et al., Pharmazie, 1985, 40(11), 782-784). The procedure how bendamustine is applied to a patient is described, e.g., in WO 2011/103150. Further parenteral bendamustine formulations have been described, e.g., in WO 2010/036702, WO 2010/097700, WO 2012/127277 and CN-A-101606934.

Despite several patent applications and scientific publications on oral formulations of bendamustine (WO 2010/063476; WO 2010/063493; WO 2010/126676; Gidwani B et al., Drug Dev Ind Pharm, 2015, 41(12), 1978-1988; Gidwani B et al., Pharm Dev Technol, 2016, 21(2), 161-171), none of the mentioned formulations for such a product are on the market.

Thus, there is still a strong and ongoing need for novel and/or improved oral formulations of bendamustine, particularly oral formulations of bendamustine that meet the criteria set out in the following.

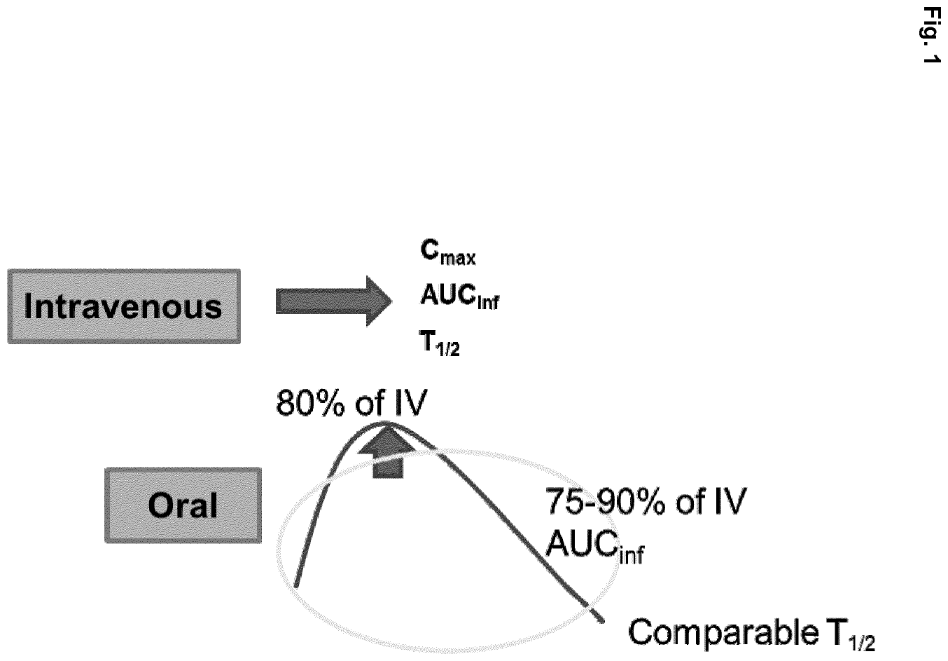

The criteria that the present inventors set for an ideal oral formulation were as follows (see also FIG. 1):
Targeted oral bioavailability for the parent drug between 75 to 90%
Targeted bioavailability for the metabolite between 120 and 140%
The half-life values for parent drug and metabolite after oral administration closely match those after intravenous administration
The appearance of metabolite after oral administration closely matches that of intravenous administration
The Cₘₐₓ after oral administration for the parent drug up to 80% of Cₘₐₓ for intravenous administration
Comparable to the intravenous (IV) formulation with respect to both Cₘₐₓ and AUC for parent drug
Formulation of metabolite reasonably comparable between oral and IV
To permit once a day dosing if bioavailability (F) is >80%
To permit twice a day dosing (BID) if bioavailability is >50% but <80%

The present invention addresses the above-discussed need. It is hence an object of the invention to provide novel and/or improved oral formulations of bendamustine, particularly oral bendamustine formulations meeting the criteria set out above.

In the context of the present invention, it has surprisingly been found that oral formulations of bendamustine with specific modified cyclodextrins, such as methyl-β-cyclodextrin, exhibit a greatly improved oral bioavailability, which renders these formulations highly advantageous for therapeutic use by oral administration, including in the treatment of cancer.

Accordingly, the present invention provides a composition for use as a medicament, wherein the composition comprises bendamustine or a pharmaceutically acceptable salt or solvate thereof in combination with a modified cyclodextrin, wherein the composition is to be administered orally, and wherein said modified cyclodextrin is selected from α-cyclodextrin, β-cyclodextrin and γ-cyclodextrin, wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention thus provides a composition for use in therapy, wherein the composition comprises bendamustine or a pharmaceutically acceptable salt or solvate thereof in combination with a modified cyclodextrin, wherein the composition is to be administered orally, and wherein said modified cyclodextrin is selected from α-cyclodextrin, β-cyclodextrin and γ-cyclodextrin, wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

In other words, the present invention provides a pharmaceutical composition for oral administration, wherein the pharmaceutical composition comprises bendamustine or a pharmaceutically acceptable salt or solvate thereof in combination with a modified cyclodextrin, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention likewise provides an oral pharmaceutical composition comprising bendamustine or a pharmaceutically acceptable salt or solvate thereof in combination with a modified cyclodextrin, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention further provides an oral pharmaceutical formulation comprising bendamustine or a pharmaceutically acceptable salt or solvate thereof in combination with a modified cyclodextrin, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention also provides a pharmaceutical composition which is formulated (or adapted) for oral administration, wherein the pharmaceutical composition comprises bendamustine or a pharmaceutically acceptable salt or solvate thereof in combination with a modified cyclodextrin, and wherein said modified cyclodextrin is selected from α-cyclodextrin, β-cyclodextrin and γ-cyclodextrin, wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

Moreover, the present invention relates to the use of bendamustine or a pharmaceutically acceptable salt or solvate thereof in combination with a modified cyclodextrin for the preparation of a medicament for oral administration, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention also relates to the use of bendamustine or a pharmaceutically acceptable salt or solvate thereof and a modified cyclodextrin for the preparation of a medicament which is formulated (or adapted) for oral administration, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The present invention furthermore refers to the use of bendamustine or a pharmaceutically acceptable salt or solvate thereof in combination with a modified cyclodextrin for the preparation of an oral medicament (or an oral pharmaceutical composition) for the treatment of a disease or disorder, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention likewise provides a method of treating a disease or disorder (e.g., cancer) in a subject/patient (e.g., a human) in need thereof, the method comprising orally administering a pharmaceutical composition comprising bendamustine or a pharmaceutically acceptable salt or solvate thereof in combination with a modified cyclodextrin to the subject/patient, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof. In particular, the method comprises the oral administration of a therapeutically effective amount of the pharmaceutical composition. Examples of the disease or disorder to be treated are described further below.

Moreover, the present invention provides a method of enhancing the oral bioavailability of bendamustine or a pharmaceutically acceptable salt or solvate thereof, the method comprising orally administering a pharmaceutical composition comprising bendamustine or a pharmaceutically acceptable salt or solvate thereof in combination with a modified cyclodextrin to a subject/patient (e.g., a human) in need thereof, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

The invention further relates to a method of delivering bendamustine or a pharmaceutically acceptable salt or solvate thereof to a subject/patient (e.g., a human) in need thereof, the method comprising orally administering a pharmaceutical composition comprising bendamustine or a pharmaceutically acceptable salt or solvate thereof in combination with a modified cyclodextrin to the subject/patient, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof. The method particularly comprises the oral administration of a therapeutically effective amount of the pharmaceutical composition.

The composition (or pharmaceutical composition, formulation or medicament) according to the present invention can be used for the treatment of various diseases or disorders, including in particular cancer (Darwish M et al., Cancer Chemother Pharmacol, 2015, 75(6), 1143-1154; Mundt M et al., Beilage zu Onkologie, Band 24, Heft 3, Juni 2001 (doi:10.1159/000055100); Cheson B D et al., J Clin Oncol, 2009, 27(9), 1492-1501), but also non-cancerous diseases/disorders (Faivre G et al., Neurology, 2014, 82(10 Supplement), P7.261 (May 1, 2014 Poster Session VII Neuro-oncology: Primary CNS Lymphoma and Other Hematologic Malignancies), http://www.neurology.org/content/82/10_Supplement/P7.261). For example, bendamustine has been shown to induce the production of interleukin-10, which suppresses inflammation, in studies with human B cells, and has thus been found to exert anti-inflammatory activity (see, e.g., Lu L et al., Int lmmunopharmacol, 2016, 39, 273-279); the composition (or pharmaceutical composition, formulation or medicament) according to the invention is hence considered to be effective in the treatment of autoimmune diseases/disorders, such as, e.g., systemic lupus erythematosus.

The cancer to be treated in accordance with the invention is preferably a hematological cancer. The hematological cancer may, for example, be selected from lymphoma, Hodgkin lymphoma, nodular sclerosing Hodgkin lymphoma, mixed-cellularity Hodgkin lymphoma, lymphocyte-rich Hodgkin lymphoma, lymphocyte-depleted Hodgkin lymphoma, nodular lymphocyte predominant Hodgkin lymphoma, non-Hodgkin lymphoma, follicular non-Hodgkin lymphoma, diffuse non-Hodgkin lymphoma, diffuse large B-cell lymphoma, Burkitt's lymphoma, mantle cell lymphoma, peripheral T-cell lymphoma, cutaneous T-cell lymphoma, mycosis fungoides, Sézary's disease, T-zone lymphoma, lymphoepithelioid lymphoma, Lennert's lymphoma, lymphosarcoma, a malignant immunoproliferative disease, Waldenström's macroglobulinemia, alpha heavy chain disease, gamma heavy chain disease, Franklin's disease, an immunoproliferative small intestinal disease, Mediterranean lymphoma, multiple myeloma, Kahler's disease, myelomatosis, leukemia, plasma cell leukemia, lymphoid leukemia, acute lymphoblastic leukemia, chronic lymphocytic leukemia, subacute lymphocytic leukemia, prolymphocytic leukemia, hairy-cell leukemia, leukemic reticuloendotheliosis, adult T-cell leukemia, myeloid leukemia, acute myeloid leukemia, chronic myeloid leukemia, subacute myeloid leukemia, myeloid sarcoma, chloroma, granulocytic sarcoma, acute promyelocytic leukemia, acute myelomonocytic leukemia, a chronic BCR-ABL negative myeloproliferative disorder, polycythaemia vera, essential thrombocythemia, idiopathic myelofibrosis, monocytic leukemia, acute erythraemia, erythroleukemia, acute erythraemic myelosis, Di Guglielmo's disease, chronic erythraemia, Heilmeyer-Schöner disease, acute megakaryoblastic leukemia, mast cell leukemia, acute panmyelosis, acute myelofibrosis, and Letterer-Siwe disease. It is particularly preferred that the cancer to be treated is a hematological cancer selected from chronic lymphocytic leukemia, Hodgkin lymphoma, non-Hodgkin lymphoma, follicular non-Hodgkin lymphoma, indolent B cell non-Hodgkin lymphoma, mantle cell lymphoma, Waldenström's macroglobulinemia, and multiple myeloma.

Moreover, the hematological cancer to be treated in accordance with the present invention, including any one of the above-mentioned specific hematological cancers (such as, e.g., chronic lymphocytic leukemia, Hodgkin lymphoma, non-Hodgkin lymphoma, follicular non-Hodgkin lymphoma, indolent B cell non-Hodgkin lymphoma, mantle cell lymphoma, Waldenström's macroglobulinemia, or multiple myeloma), may be a relapsed or refractory hematological cancer (e.g., a hematological cancer refractory to alkylating agents), preferably a rituximab-refractory hematological cancer.

The cancer to be treated in accordance with the present invention may also be a solid cancer. In particular, the cancer to be treated may be selected from breast cancer (e.g., metastatic breast cancer, particularly pretreated metastatic or advanced breast cancer), lung cancer (particularly small-cell lung cancer), ovarian cancer, colorectal cancer, colon cancer, pancreatic cancer, bladder cancer, prostate cancer, head and/or neck cancer, and soft-tissue sarcoma.

As explained above, the composition (or pharmaceutical composition) according to the invention can also be used for the treatment of diseases or disorders other than cancer. For example, the disease or disorder to be treated in accordance with the present invention may also be an autoimmune disease/disorder, e.g., rheumatoid arthritis, multiple sclerosis (such as, e.g., relapsing-remitting multiple sclerosis, primary progressive multiple sclerosis, secondary progressive multiple sclerosis, clinically isolated syndrome, Devic's disease, Balo concentric sclerosis, Schilder's diffuse sclerosis, or Marburg multiple sclerosis), or lupus erythematosus (such as, e.g., systemic lupus erythematosus, acute cutaneous lupus erythematosus, subacute cutaneous lupus erythematosus, discoid lupus erythematosus, chilblain lupus erythematosus, lupus erythematosus-lichen planus overlap syndrome, lupus erythematosus panniculitis, tumid lupus erythematosus, verrucous lupus erythematosus, cutaneous lupus mucinosis, drug-induced lupus erythematosus, or neonatal lupus erythematosus; particularly systemic lupus erythematosus). The disease or disorder to be treated in accordance with the invention may also be a neurodegenerative disease/disorder, e.g., Parkinson's disease, Alzheimer's disease, or Huntington's disease. Moreover, the composition (or pharmaceutical composition) according to the invention can also be used in immunomodulatory therapy, i.e. as an immunomodulatory therapeutic agent.

The present invention is also described by the appended illustrative figures which show:

FIG. 1: Pharmacokinetic profile and parameters of oral vs. intravenous (IV) administration.

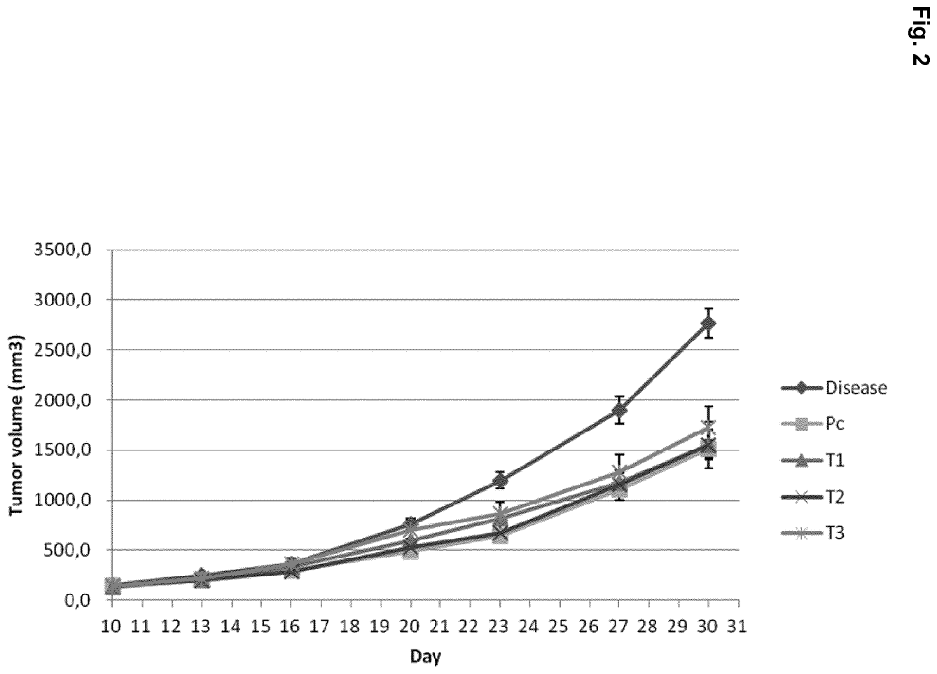

FIG. 2: Tumor volume in a Burkitt cancer cell xenograft model in NOD/Scid mice: Disease=Control; Pc=Bendamustine i.v. (25 mg/kg); T1=Bendamustine-Me-β-CD Formulation oral (30 mg/kg); T2=Bendamustine-2-HP-β-CD Formulation oral (30 mg/kg); T3=Bendamustine in water oral. See Example 9.

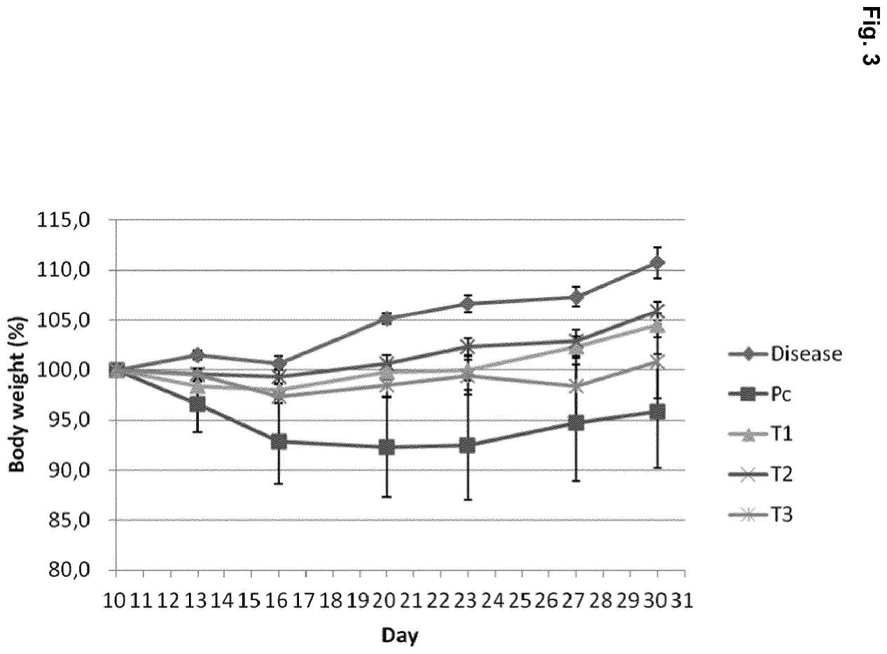

FIG. 3: Body weight in a Burkitt cancer cell xenograft model in NOD/Scid mice: Disease=Control; Pc=Bendamustine i.v. (25 mg/kg); T1=Bendamustine-Me-β-CD Formulation oral (30 mg/kg); T2=Bendamustine-2-HP-β-CD Formulation oral (30 mg/kg); T3=Bendamustine in water oral. See Example 9.

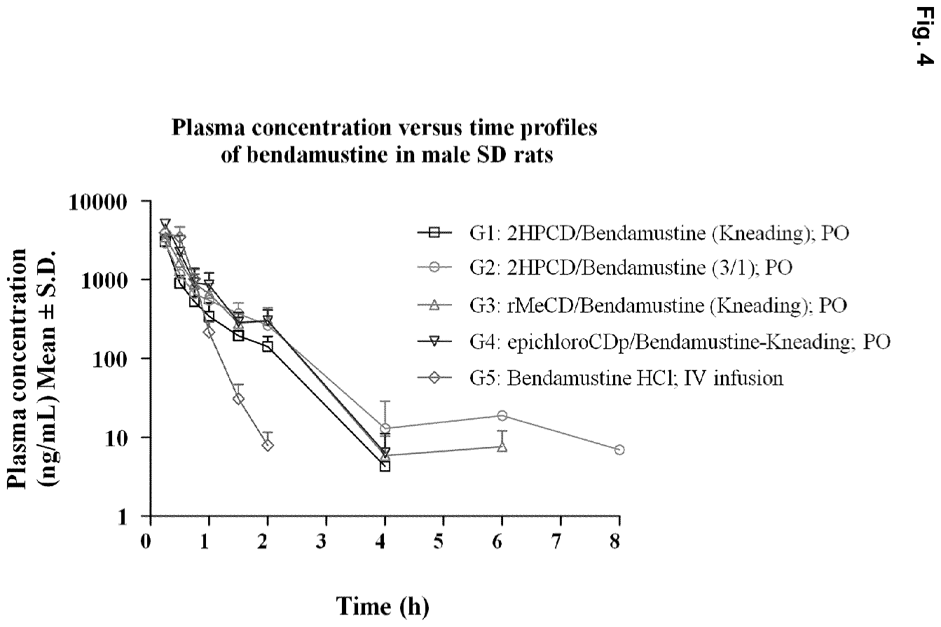

FIG. 4: Plasma concentration versus time profiles of bendamustine formulations with 2-hydroxypropyl-β-cyclodextrin (2HPCD), randomized methyl-β-cyclodextrin (rMeCD), and polymerized epichlorohydrin-ß-cyclodextrin (epichloroCDp; reference), respectively, in male SD rats. See Example 11.

The following detailed description applies to all of the aspects and embodiments of the present invention as described and defined herein above.

As described above, the composition (or pharmaceutical composition or formulation) provided in accordance with the present invention comprises bendamustine or a pharmaceutically acceptable salt or solvate thereof. Preferably, it comprises bendamustin hydrochloride. More preferably, it comprises bendamustin hydrochloride monohydrate. Bendamustine, bendamustin hydrochloride and bendamustin hydrochloride monohydrate are known in the art and have been described, e.g., in the Chemical Abstracts Services (CAS) registry, particularly under CAS nos. 16506-27-7, 3543-75-7 and 1374784-02-7, respectively.

The composition (or pharmaceutical composition/formulation) provided in accordance with the invention further comprises a modified cyclodextrin which is selected from α-cyclodextrin, β-cyclodextrin and γ-cyclodextrin, wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof. The modified cyclodextrin may thus be an α-, β- or γ-cyclodextrin which is substituted with one or more groups selected independently from C₁₋₄ alkyl, hydroxy-C₁₋₄ alkyl, dihydroxy-C₁₋₄ alkyl, and —CO(C₁₋₄ alkyl).

α-Cyclodextrin (α-CD), β-cyclodextrin (β-CD) and γ-cyclodextrin (γ-CD) are composed of six (in the case of α-CD), seven (in the case of β-CD) and eight (in the case of γ-CD) α-D-glucose monomer units (which may also be referred to as anhydroglucose units), respectively, which are connected via α-1,4-glycosidic bonds to form a cyclic oligosaccharide. In the modified cyclodextrins to be used in the present invention, one or more of the free hydroxy groups in positions 2, 3 and/or 6 of the glucose units of α-CD, β-CD or γ-CD are substituted. Depending on how many hydroxy groups (and how many glucose units) of a cyclodextrin are substituted in this way, modified cyclodextrins with different degrees of substitution can be obtained. The total degree of substitution (TDS), which indicates the average number of substituent groups per cyclodextrin molecule, can be determined, e.g., as described in: Challa R et al., AAPS PharmSciTech, 2005, 6(2), E329-E357; Choisnard L et al., Biomacromolecules, 2011, 12(8), 3031-3038; or Yuan C et al., Journal of Investigative Medicine, 2014, 62(8 Suppl), S107. In the case of β-cyclodextrins, the total degree of substitution (TDS) is also referred to as MS₇ value (Roquette, “Kleptose® Betacyclodextrins & Hydroxypropyl Betacyclodextrins”, manufacturer's brochure, 2006).

Preferably, the modified cyclodextrin to be used in the present invention is a modified β-cyclodextrin, i.e., β-cyclodextrin substituted with C₁₋₄ alkyl, hydroxy-C₁₋₄ alkyl, dihydroxy-C₁₋₄ alkyl, —CO(C₁₋₄ alkyl), or any combination thereof. The modified β-cyclodextrin may, for example, have a total degree of substitution (TDS or MS₇ value) of about 2 to about 16, particularly of about 3 to about 14 (e.g., about 3, about 4, about 5, about 6, about 7, about 8, about 9, about 10, about 11, about 12, about 13, or about 14).

The substituent(s) on the modified cyclodextrin (e.g., on the modified β-cyclodextrin) is/are selected from C₁₋₄ alkyl, hydroxy-C₁₋₄ alkyl, dihydroxy-C₁₋₄ alkyl, —CO(C₁₋₄ alkyl), and any combination thereof. Preferably, the substituent(s) is/are selected from methyl, hydroxyethyl (e.g., 1-hydroxyethyl or 2-hydroxyethyl; particularly 2-hydroxyethyl), hydroxypropyl (e.g., 1-hydroxypropyl, 2-hydroxypropyl, β-hydroxypropyl, 1-hydroxy-1-methylethyl or 2-hydroxy-1-methylethyl; particularly 2-hydroxypropyl or β-hydroxypropyl; more preferably 2-hydroxypropyl), dihydroxypropyl (e.g., 1,1-dihydroxypropyl, 2,2-dihydroxypropyl, 3,3-dihydroxypropyl, 1,2-dihydroxypropyl, 1,3-dihydroxypropyl, 2,3-dihydroxypropyl, 2,2-dihydroxy-1-methylethyl, 1,2-dihydroxy-1-methylethyl or 1-(hydroxymethyl)-2-hydroxyethyl; such as 2,3-dihydroxypropyl), hydroxybutyl (e.g., 1-hydroxybutyl, 2-hydroxybutyl, 3-hydroxybutyl, or 4-hydroxybutyl; such as 2-hydroxybutyl), acetyl (i.e., —C(═O)—CH₃), and any combination thereof.

It is thus particularly preferred that the modified cyclodextrin is β-cyclodextrin which is substituted with methyl, hydroxyethyl, hydroxypropyl, dihydroxypropyl, hydroxybutyl, acetyl, or any combination thereof. Accordingly, it is particularly preferred that the modified cyclodextrin is selected from methyl-β-cyclodextrin, hydroxypropyl-β-cyclodextrin (e.g., (2-hydroxypropyl)-β-CD), hydroxyethyl-β-cyclodextrin (e.g., (2-hydroxyethyl)-β-CD), dihydroxypropyl-β-cyclodextrin (e.g., (2,3-dihydroxypropyl)-β-CD), hydroxybutyl-β-cyclodextrin (e.g., (2-hydroxybutyl)-β-CD), acetyl-β-cyclodextrin, and a β-cyclodextrin substituted with at least two different groups selected from methyl, hydroxyethyl, hydroxypropyl, dihydroxypropyl, hydroxybutyl, and acetyl (e.g., a β-cyclodextrin substituted with methyl and acetyl, such as heptakis(3-O-acetyl-2,6-di-O-methyl)-β-cyclodextrin).

Even more preferably, the modified cyclodextrin is methyl-β-cyclodextrin or hydroxypropyl-β-cyclodextrin. The methyl-β-cyclodextrin may be, e.g., 2-O-methyl-β-cyclodextrin, 3-O-methyl-β-cyclodextrin, 6-O-methyl-β-cyclodextrin, 2,3-di-O-methyl-β-cyclodextrin, 2,6-di-O-methyl-β-cyclodextrin, 3,6-di-O-methyl-β-cyclodextrin, 2,3,6-tri-O-methyl-β-cyclodextrin, or random methyl-β-cyclodextrin (i.e., randomly methylated β-cyclodextrin); it is preferably random methyl-β-cyclodextrin, heptakis(2,6-di-O-methyl)-β-cyclodextrin or heptakis(2,3,6-tri-O-methyl)-β-cyclodextrin, and is more preferably random methyl-β-cyclodextrin or heptakis(2,6-di-O-methyl)-β-cyclodextrin. A corresponding exemplary random methyl-β-cyclodextrin may, for instance, have a molecular weight of about 1310 Da and/or may contain, on average, about 1.6 to about 2.0 methyl groups per anhydroglucose unit of the β-cyclodextrin. The hydroxypropyl-β-cyclodextrin may be, e.g., 2-O-(2-hydroxypropyl)-β-cyclodextrin, 3-O-(2-hydroxypropyl)-β-cyclodextrin, 6-O-(2-hydroxypropyl)-β-cyclodextrin, 2,3-d i-O-(2-hydroxypropyl)-β-cyclodextrin, 2,6-d i-O-(2-hydroxypropyl)-β-cyclodextrin, 3,6-di-O-(2-hydroxypropyl)-β-cyclodextrin, 2,3,6-tri-O-(2-hydroxypropyl)-β-cyclodextrin, or random hydroxypropyl-β-cyclodextrin, and it is preferably random hydroxypropyl-β-cyclodextrin (particularly random hydroxypropyl-β-cyclodextrin having an MS₇ value of about 4 to about 6, e.g., about 4.5 or about 5.6). A corresponding exemplary random hydroxypropyl-β-cyclodextrin may, for instance, have a molecular weight of about 1540 Da. Most preferably, the modified cyclodextrin is methyl-β-cyclodextrin (particularly random methyl-β-cyclodextrin or heptakis(2,6-di-O-methyl)-β-cyclodextrin).

The composition (or pharmaceutical composition) according to the present invention may comprise one single type of modified cyclodextrin (e.g., only methyl-β-cyclodextrin), or it may comprise a mixture of two or more different types of modified cyclodextrins (e.g., a mixture of methyl-β-cyclodextrin and hydroxypropyl-β-cyclodextrin, which may be present in the composition, for example, in a molar ratio in the range of about 1:10 to about 10:1, particularly in a molar ratio of about 1:1). It is preferred that the composition comprises a single type of modified cyclodextrin (which is preferably methyl-β-cyclodextrin).

As described above, the composition (or pharmaceutical composition) provided in accordance with the present invention comprises bendamustine or a pharmaceutically acceptable salt or solvate thereof in combination with a modified cyclodextrin. In particular, it comprises an inclusion complex of said modified cyclodextrin and said bendamustine or the pharmaceutically acceptable salt or solvate thereof. Inclusion complexes of bendamustine (or a pharmaceutically acceptable salt or solvate thereof) and the modified cyclodextrin can be prepared using methods known in the art for the formation of cyclodextrin inclusion complexes, including, e.g., kneading, physically mixing, co-evaporating, freeze-drying, or spray-drying. Such techniques, which can be used to form inclusion complexes of the modified cyclodextrin and bendamustine in accordance with the present invention, are further described in the literature, e.g., in: Del Valle EMM, Process biochemistry, 2004, 39(9), 1033-1046; Nasir A et al., Int Res J Pharm, 2012, 3(4), 44-50; Loftsson T et al., J Pharm Sci, 1996, 85, 1017-1025; Roquette, “Kleptose® Betacyclodextrins & Hydroxypropyl Betacyclodextrins”, manufacturer's brochure, 2006; Gidwani B et al., Drug Dev Ind Pharm, 2015, 41(12), 1978-1988; Blanco J et al., Drug development and industrial pharmacy, 1991, 17(7), 943-957; or Junco S et al., Journal of inclusion phenomena and macrocyclic chemistry, 2002, 44(1-4), 117-121. The inclusion complex of the modified cyclodextrin and the bendamustine (or the pharmaceutically acceptable salt or solvate thereof) comprised in the composition (or pharmaceutical composition) of the present invention can thus be obtained (i.e., is obtainable), e.g., by kneading, physically mixing, co-evaporating, freeze-drying, or spray-drying the modified cyclodextrin and the bendamustine (or the pharmaceutically acceptable salt or solvate thereof). Preferably, the inclusion complex is obtained/obtainable by kneading the modified cyclodextrin and the bendamustine (or the pharmaceutically acceptable salt or solvate thereof).

Bendamustine (or a pharmaceutically acceptable salt or solvate thereof) and the modified cyclodextrin are preferably comprised in the composition (or the pharmaceutical composition) according to the invention ata molar ratio of about 1:10 to about 1:0.5 (e.g. at a molar ratio of about 1:0.5, about 1:0.6, about 1:0.8, about 1:1, about 1:1.5, about 1:2, or about 1:3), more preferably at a molar ratio of about 1:10 to about 1:1, even more preferably at a molar ratio of about 1:10 to about 1:1.5, yet even more preferably ata molar ratio of about 1:10 to about 1:2, and still more preferably at a molar ratio of about 1:10 to about 1:3.

The scope of the invention embraces all pharmaceutically acceptable salt forms of the compounds provided herein, particularly of bendamustine, which may be formed, e.g., by protonation of an atom carrying an electron lone pair which is susceptible to protonation, such as an amino group, with an inorganic or organic acid, or as a salt of an acid group, such as a carboxylic acid group, with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred examples of pharmaceutically acceptable salts of bendamustine include, in particular, a hydrochloride salt, a hydrobromide salt, a mesylate salt, a sulfate salt, a tartrate salt, a fumarate salt, an acetate salt, a citrate salt, or a phosphate salt. A particularly preferred pharmaceutically acceptable salt of bendamustine is the hydrochloride salt.

Moreover, the scope of the invention embraces the compounds provided herein, particularly bendamustine, in any solvated form, including, e.g., solvates with water (i.e., as a hydrate) or solvates with organic solvents such as, e.g., methanol, ethanol or acetonitrile (i.e., as a methanolate, ethanolate or acetonitrilate), or in any crystalline form (i.e., as any polymorph), or in amorphous form. It is to be understood that such solvates also include solvates of pharmaceutically acceptable salts of the respective compounds (e.g., a hydrate of bendamustine hydrochloride, particularly bendamustine hydrochloride monohydrate).

As explained above, the bendamustine to be used in accordance with the present invention may be in any crystalline form (polymorph) or in amorphous form. Various polymorphs of bendamustine (particularly of bendamustine hydrochloride) as well as amorphous bendamustine have been described in the literature, e.g., in WO 2010/144675, WO 2009/120386, U.S. Pat. No. 8,445,524, or CN-A-102351799. Any one of these forms of bendamustine can be used in accordance with the present invention.

For example, the composition (or pharmaceutical composition or formulation) of the invention may comprise:
(i) crystalline bendamustine hydrochloride which is characterized by having an X-ray powder diffraction (XRPD) pattern comprising peaks at 3.3, 11.1, 12.0, 16.0 and 16.6 degrees 2-theta; or
(ii) crystalline bendamustine hydrochloride which is characterized by having an XRPD pattern comprising peaks at 14.1, 22.0, 22.9, 24.9 and 25.1 degrees 2-theta; or
(iii) crystalline bendamustine hydrochloride which is characterized by having an XRPD pattern comprising peaks at 14.1, 16.8, 17.5, 18.5, 22.0, 22.9, 24.9, 25.1 and 28.3 degrees 2-theta; or
(iv) crystalline bendamustine hydrochloride which is characterized by having an XRPD pattern comprising peaks at 26.1, 27.9 and 28.1 degrees 2-theta; or
(v) crystalline bendamustine hydrochloride which is characterized by having an XRPD pattern comprising peaks at 10.6, 15.6, 19.8, 26.1, 27.9 and 28.1 degrees 2-theta; or
(vi) crystalline bendamustine hydrochloride which is characterized by having an XRPD pattern comprising peaks at 10.8, 15.5, 20.5 and 23.6 degrees 2-theta; or
(vii) crystalline bendamustine hydrochloride which is characterized by having an XRPD pattern comprising peaks at 10.3, 10.8, 15.5, 19.6, 20.5, 20.7, 21.2, 23.6, 25.8 and 27.6 degrees 2-theta; or
(viii) crystalline bendamustine hydrochloride which is characterized by having an XRPD pattern comprising peaks at 8.3, 16.8 and 18.5 degrees 2-theta; or
(ix) crystalline bendamustine hydrochloride which is characterized by having an XRPD pattern comprising peaks at 8.3, 14.0, 16.8 and 18.5 degrees 2-theta; or
(x) crystalline bendamustine hydrochloride which is characterized by having an XRPD pattern comprising peaks at 8.3, 14.0, 16.8, 18.5, 22.0, 22.9, 25.1 and 28.3 degrees 2-theta; or
(xi) crystalline bendamustine hydrochloride which is characterized by having an XRPD pattern comprising peaks at 10.6, 15.0, 18.7, 20.0, 22.9 and 26.5 degrees 2-theta; or
(xii) crystalline bendamustine hydrochloride which is characterized by having an XRPD pattern comprising peaks at 7.4, 10.6, 13.6, 15.0, 17.4, 18.7, 20.0, 20.3, 22.0, 22.9, 24.3 and 26.5 degrees 2-theta; or
(xiii) bendamustine hydrochloride Form 1 (as described in WO 2009/120386); or
(xiv) bendamustine hydrochloride Form 3 (as described in WO 2009/120386); or
(xv) bendamustine hydrochloride Form 4 (as described in WO 2009/120386); or
(xvi) a mixture of any two or more of the crystalline forms listed in items (i) to (xv) above.

It is to be understood that each of the XRPD peaks referred to in items (i) to (xii) above has the indicated numerical value±0.2 degrees 2-theta, preferably±0.1 degrees 2-theta, and even more preferably has the exact numerical value indicated. The above-mentioned crystalline forms of bendamustine hydrochloride can be prepared, e.g., using the procedures described in WO 2010/144675, WO 2009/120386, U.S. Pat. No. 8,445,524 or CN-A-102351799.

The present invention also encompasses the use of prodrugs, particularly the use of a pharmaceutically acceptable prodrug of bendamustine (which can be employed in place of bendamustine). Prodrugs are derivatives of pharmaceutically active parent compounds, which have chemically or metabolically cleavable groups and are converted, by solvolysis or under physiological conditions, into the respective pharmaceutically active parent compound. Prodrugs include acid derivatives, such as, e.g., esters prepared by reaction of the parent acidic compound with a suitable alcohol, or amides prepared by reaction of the parent acidic compound with a suitable amine. If a compound to be used in the present invention has a carboxyl group (e.g., bendamustine), an ester derivative prepared by reacting the carboxyl group with a suitable alcohol or an amide derivative prepared by reacting the carboxyl group with a suitable amine is exemplified as a prodrug. Corresponding exemplary ester derivatives (e.g., of bendamustine) which can be used as prodrugs include, in particular, methyl ester, ethyl ester, n-propyl ester, isopropyl ester, n-butyl ester, isobutyl ester, tert-butyl ester, morpholinoethyl ester, or α-acetoxyethyl ester. If a compound to be used in the present invention has a hydroxy group, an acyloxy derivative prepared by reacting the hydroxy group with an suitable acyl halide or a suitable acid anhydride is exemplified as a prodrug. If a compound to be used in the present invention has an amino group, an amide derivative prepared by reacting the amino group with a suitable acid halide or a suitable mixed anhydride is exemplified as a prodrug. Corresponding exemplary amide derivatives which can be used as prodrugs are —NHC(═O)—(CH₂)₂OCH₃ or —NHC(═O)—CH(NH₂)CH₃. Thus, also a prodrug of bendamustine can be used, in which the carboxyl group of bendamustine is in the form of an ester or in the form of an amide, particularly in the form of an ester (such as, e.g., a methyl ester, an ethyl ester, an n-propyl ester, an isopropyl ester, an n-butyl ester, an isobutyl ester, a tert-butyl ester, a morpholinoethyl ester, or an α-acetoxyethyl ester of bendamustine). Corresponding examples of such prodrugs of bendamustine, such as, e.g., bendamustine methyl ester, bendamustine ethyl ester, bendamustine propyl ester, bendamustine isopropyl ester, bendamustine butyl ester, bendamustine morpholinoethyl ester, bendamustine piperidinoethyl ester, bendamustine pyrrolidinoethyl ester, or bendamustine methylpiperazinoethyl ester, are further described in the literature, e.g., in EP-A-2656843. The present invention also embraces compositions as described and defined herein, which contain any one of the aforementioned prodrugs of bendamustine or any one of the compounds disclosed in EP-A-2656843 in place of bendamustine.

The composition provided in accordance with the present invention is preferably a pharmaceutical composition. Pharmaceutical compositions can be formulated by techniques known in the art, including in particular the techniques described in “Remington: The Science and Practice of Pharmacy”, Pharmaceutical Press, 22^{nd} edition. The pharmaceutical compositions according to the invention are formulated for oral administration. They optionally comprise one or more pharmaceutically acceptable excipients, such as, e.g., carriers, fillers, disintegrants, lubricating agents, binders, colorants, pigments, stabilizers, preservatives, antioxidants, sweetening agents, and/or flavoring agents. In particular, the pharmaceutical compositions may contain one or more pharmaceutically acceptable excipients such as non-reducing sugars, microcrystalline cellulose, sodium citrate, calcium carbonate, dibasic calcium phosphate or glycine, disintegrants such as starch (e.g., corn, potato or tapioca starch), sodium starch glycolate, croscarmellose sodium or complex silicates, granulation binders such as polyvinylpyrrolidone, hydroxypropylmethylcellulose (HPMC), hydroxypropylcellulose (HPC), sucrose or acacia, lubricating agents such as magnesium stearate, stearic acid, glyceryl behenate or talc, and/or enhancers such as sodium N-[8-(2-hydroxybenzoyl)amino]caprylate (“SNAC”).

The composition (or pharmaceutical composition) according to the present invention is for oral administration, i.e., it is intended to be administered orally, particularly via peroral ingestion or swallowing.

The composition (or pharmaceutical composition) according to the present invention can be provided in any form, particularly in any pharmaceutical dosage form, that is suitable for oral administration, including as a solid composition or as a liquid composition. Dosage forms for oral administration include, e.g., pills, tablets (e.g., chewing tablets or effervescent tablets), mini-tablets, capsules, lozenges, troches, pellets, ovules, solutions, emulsions, suspensions, syrups, elixirs, powders, granules, films, medicated gums, and multiparticulate dosage forms.

It is preferred that the composition (or pharmaceutical composition) according to the invention is a solid composition, particularly that it is provided in the form of a solid oral dosage form. Preferred examples of a solid oral dosage form include a pill, a tablet, a mini-tablet, a capsule (e.g., a gelatin capsule, an HPMC capsule [e.g., a Vcaps® Plus HPMC capsule, as available, e.g., from Capsugel], or a PVP capsule), a lozenge, a troche, a pellet, a powder, granules, or a film. The solid composition (or the solid oral dosage form, including any of the aforementioned exemplary solid oral dosage forms) is not particularly limited with respect to its water content, as long as it is in solid form. For example, the solid composition (or the solid oral dosage form) may contain less than about 15% (w/w) of water, preferably less than about 10% (w/w) of water, more preferably less than about 5% (w/w) of water. Such solid compositions (or solid oral dosage forms) having a low content of water are advantageous as they provide an improved shelf-stability and thus enable prolonged storage periods.

The solid oral dosage form, which is preferably a pill, a tablet, a mini-tablet, a capsule, a lozenge, a troche, a pellet, a powder, granules, or a film, may furthermore have an enteric coating. Such coatings are known in the art and are not particularly limited. For example, the enteric coating may be made from a material selected from methyl acrylate-methacrylic acid copolymer, ethyl acrylate-methylacrylic acid copolymer, methyl methacrylate-methacrylic acid copolymer, cellulose acetate phthalate, cellulose acetate succinate, hydroxypropyl methyl cellulose, hydroxypropyl methyl cellulose phthalate, hydroxypropyl methyl cellulose acetate succinate, polyvinyl acetate phthalate, shellac, cellulose acetate trimellitate, carboxymethyl cellulose, sodium alginate, zein, amylose, starch, and dextrins. Further details about these coatings as well as other suitable enteric coatings can be found in the literature, e.g., in: Wen H et al., Oral controlled release formulation design and drug delivery: theory to practice, John Wiley & Sons, 2011; Aulton M et al., Pharmaceutical coating technology, Taylor & Francis, 1995; or Hussan S D et al., IOSR Journal of Pharmacy, 2012, 2(6), 5-11.

While the composition according to the invention is preferably a solid composition, as explained above, it is also possible that the composition according to the invention may be a liquid composition. In that case, it is advantageous to use a liquid composition that contains less than about 15% (w/w) of water, preferably less than about 10% (w/w) of water, more preferably less than about 5% (w/w) of water.

Moreover, it is also possible, although not preferred, that the composition according to the invention is an aqueous liquid composition (e.g., an aqueous solution). In this case, the composition should preferably be prepared shortly before administration to the subject/patient, and prolonged storage periods should be avoided.

Typically, a physician will determine the actual dosage which will be most suitable for an individual subject. The specific dose level and frequency of dosage for any particular individual subject may be varied and will depend upon a variety of factors including age, body weight, general health, sex, diet, and the severity of the particular condition of the individual subject undergoing therapy.

A proposed, yet non-limiting dose of the composition according to the invention for oral administration to a human subject (e.g., a human of about 70 kg body weight) contains about 10 mg to about 1 g, preferably about 20 mg to about 800 mg, more preferably about 30 mg to about 600 mg, even more preferably about 50 mg to about 500 mg (e.g., about 50 mg, about 75 mg, about 100 mg, about 150 mg, about 180 mg, about 200 mg, about 250 mg, about 300 mg, about 350 mg, about 400 mg, about 450 mg, or about 500 mg), of the active ingredient (i.e., bendamustine or a pharmaceutically acceptable salt or solvate thereof) per unit dose. The unit dose may be administered, e.g., one to five times every three or four weeks. In particular, the composition according to the invention may be administered orally to a human subject in a unit dose of about 50 mg to about 500 mg (e.g., about 50 mg, about 75 mg, about 100 mg, about 150 mg, about 180 mg, about 200 mg, about 250 mg, about 300 mg, about 350 mg, about 400 mg, about 450 mg, or about 500 mg), wherein the unit dose is to be administered: (i) on days 1 and 2 every 21 days; or (ii) on days 1 to 5 every 21 days; or (iii) on days 1 and 2 every 28 days; or (iv) on days 1 and 15 every 28 days; or (v) on days 1 to 5 every 28 days. It will be understood that the doses specified in this paragraph refer to the amount of bendamustine or a pharmaceutically acceptable salt or solvate thereof (such as bendamustine hydrochloride monohydrate) that corresponds to the indicated mass of bendamustine in non-salt form. It will further be appreciated that it may be necessary to make routine variations to the dosage depending on the age and weight of the patient/subject as well as the severity of the condition to be treated. The precise dose will ultimately be at the discretion of the attending physician.

The composition (or pharmaceutical composition) according to the invention may comprise bendamustine or a pharmaceutically acceptable salt or solvate thereof as the sole pharmaceutically active ingredient. The corresponding composition can be administered in monotherapy, e.g., without concomitantly administering any further therapeutic agents, or without concomitantly administering any further therapeutic agents against the same disease that is to be treated with bendamustine or the pharmaceutically acceptable salt or solvate thereof.

However, the composition (or pharmaceutical composition) comprising bendamustine or a pharmaceutically acceptable salt or solvate thereof in accordance with the present invention can also be administered in combination with one or more further therapeutic agents. If bendamustine (or a pharmaceutically acceptable salt or solvate thereof) is used in combination with a second therapeutic agent active against the same disease or condition, the dose of each compound may differ from that when the corresponding compound is used alone, in particular, a lower dose of each compound may be used. The combination of bendamustine (or a pharmaceutically acceptable salt or solvate thereof) with one or more further therapeutic agents may comprise the simultaneous/concomitant administration of bendamustine (or the pharmaceutically acceptable salt or solvate thereof) and the further therapeutic agent(s), either in a single pharmaceutical formulation or in separate pharmaceutical formulations, or the sequential/separate administration of bendamustine (or the pharmaceutically acceptable salt or solvate thereof) and the further therapeutic agent(s). If administration is sequential, either bendamustine (or the pharmaceutically acceptable salt or solvate thereof) or the one or more further therapeutic agents may be administered first. If administration is simultaneous, the one or more further therapeutic agents may be included in the same pharmaceutical formulation as bendamustine (or the pharmaceutically acceptable salt or solvate thereof), or they may be administered in one or more different (separate) pharmaceutical formulations.

If the composition according to the invention is used for the treatment of cancer, it is preferred that the one or more further therapeutic agents to be administered in combination with bendamustine (or the pharmaceutically acceptable salt or solvate thereof) are anticancer drugs (i.e., anticancer agents). The anticancer drug(s) to be administered in combination with bendamustine (or the pharmaceutically acceptable salt or solvate thereof) may, e.g., be selected from: a tumor angiogenesis inhibitor (e.g., a protease inhibitor, an epidermal growth factor receptor kinase inhibitor, or a vascular endothelial growth factor receptor kinase inhibitor); a cytotoxic drug (e.g., an antimetabolite, such as purine and pyrimidine analog antimetabolites); an antimitotic agent (e.g., a microtubule stabilizing drug or an antimitotic alkaloid); a platinum coordination complex; an anti-tumor antibiotic; an alkylating agent (e.g., a nitrogen mustard or a nitrosourea); an endocrine agent (e.g., an adrenocorticosteroid, an androgen, an anti-androgen, an estrogen, an anti-estrogen, an aromatase inhibitor, a gonadotropin-releasing hormone agonist, or a somatostatin analog); or a compound that targets an enzyme or receptor that is overexpressed and/or otherwise involved in a specific metabolic pathway that is deregulated (or misregulated) in the tumor cell (e.g., ATP and GTP phosphodiesterase inhibitors, histone deacetylase inhibitors, protein kinase inhibitors (such as serine, threonine and tyrosine kinase inhibitors, e.g., Abelson protein tyrosine kinase inhibitors) and the various growth factors, their receptors and corresponding kinase inhibitors (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (e.g., cyclooxygenase-1 or cyclooxygenase-2 inhibitors), topoisomerase inhibitors (e.g., topoisomerase I inhibitors or topoisomerase II inhibitors), poly ADP ribose polymerase inhibitors (PARP inhibitors), and epidermal growth factor receptor (EGFR) inhibitors/antagonists.

An alkylating agent which can be used as an anticancer drug in combination with bendamustine (or the pharmaceutically acceptable salt or solvate thereof) may be, for example, a nitrogen mustard (such as cyclophosphamide, mechlorethamine (chlormethine), uramustine, melphalan, chlorambucil, ifosfamide, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazine (such as temozolomide).

A platinum coordination complex which can be used as an anticancer drug in combination with bendamustine (or the pharmaceutically acceptable salt or solvate thereof) may be, for example, cisplatin, carboplatin, nedaplatin, oxaliplatin, satraplatin, or triplatin tetranitrate.

A cytotoxic drug which can be used as an anticancer drug in combination with bendamustine (or the pharmaceutically acceptable salt or solvate thereof) may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with bendamustine (or the pharmaceutically acceptable salt or solvate thereof) may be, for example, a taxane (such as docetaxel, larotaxel, ortataxel, paclitaxel/taxol, tesetaxel, or nab-paclitaxel (e.g., Abraxane®)), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with bendamustine (or the pharmaceutically acceptable salt or solvate thereof) may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin).

A tyrosine kinase inhibitor which can be used as an anticancer drug in combination with bendamustine (or the pharmaceutically acceptable salt or solvate thereof) may be, for example, axitinib, bosutinib, cediranib, dasatinib, erlotinib, gefitinib, imatinib, lapatinib, lestaurtinib, nilotinib, semaxanib, sorafenib, sunitinib, axitinib, nintedanib, ponatinib, or vandetanib.

A topoisomerase inhibitor which can be used as an anticancer drug in combination with bendamustine (or the pharmaceutically acceptable salt or solvate thereof) may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

A PARP inhibitor which can be used as an anticancer drug in combination with bendamustine (or the pharmaceutically acceptable salt or solvate thereof) may be, for example, BMN-673, olaparib, rucaparib, veliparib, CEP 9722, MK 4827, BGB-290, or 3-aminobenzamide.

An EGFR inhibitor/antagonist which can be used as an anticancer drug in combination with bendamustine (or the pharmaceutically acceptable salt or solvate thereof) may be, for example, gefitinib, erlotinib, lapatinib, afatinib, neratinib, osimertinib, ABT-414, dacomitinib, AV-412, PD 153035, vandetanib, PKI-166, pelitinib, canertinib, icotinib, poziotinib, BMS-690514, CUDC-101, AP26113, XL647, cetuximab, panitumumab, zalutumumab, nimotuzumab, or matuzumab.

Further anticancer drugs may also be used in combination with bendamustine (or the pharmaceutically acceptable salt or solvate thereof). The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, iniparib, or copanlisib.

Also biological drugs, like antibodies, antibody fragments, antibody constructs (for example, single-chain constructs), and/or modified antibodies (like CDR-grafted antibodies, humanized antibodies, “full humanized” antibodies, antibody-drug conjugates, etc.) directed against cancer or tumor markers/factors/cytokines involved in proliferative diseases can be employed in co-therapeutic approaches with bendamustine (or the pharmaceutically acceptable salt or solvate thereof) in accordance with the present invention. Examples of such biological molecules are anti-HER2 antibodies (e.g., trastuzumab), anti-CD20 antibodies (e.g., rituximab, ocrelizumab, ofatumumab, obinutuzumab, or ibritumomab tiuxetan), anti-CD19/CD3 constructs, trastuzumab emtansin, brentuximab vedotin, or anti-TNF antibodies.

An anticancer drug which can be used in combination with bendamustine (or the pharmaceutically acceptable salt or solvate thereof) may also be an immunooncology therapeutic (such as an antibody (e.g., a monoclonal antibody or a polyclonal antibody), an antibody fragment, an antibody construct (e.g., a single-chain construct), or a modified antibody (e.g., a CDR-grafted antibody, a humanized antibody, or a “full humanized” antibody) targeting any one of CTLA-4, PD-1/PD-L1, TIM3, LAG3, OX4, CSF1R, IDO, or CD40. Such immunooncology therapeutics include, e.g., an anti-CTLA-4 antibody (particularly an antagonistic or pathway-blocking anti-CTLA-4 antibody; e.g., ipilimumab or tremelimumab), an anti-PD-1 antibody (particularly an antagonistic or pathway-blocking anti-PD-1 antibody; e.g., nivolumab (BMS-936558), pembrolizumab (MK-3475), pidilizumab (CT-011), AMP-224, or APE02058), an anti-PD-L1 antibody (particularly a pathway-blocking anti-PD-L1 antibody; e.g., BMS-936559, MEDI4736, MPDL3280A (RG7446), MDX-1105, or MEDI6469), an anti-TIM3 antibody (particularly a pathway-blocking anti-TIM3 antibody), an anti-LAG3 antibody (particularly an antagonistic or pathway-blocking anti-LAG3 antibody; e.g., BMS-986016, IMP701, or IMP731), an anti-OX4 antibody (particularly an agonistic anti-OX4 antibody; e.g., MEDI0562), an anti-CSF1R antibody (particularly a pathway-blocking anti-CSF1R antibody; e.g., IMC-CS4 or RG7155), an anti-IDO antibody (particularly a pathway-blocking anti-IDO antibody), or an anti-CD40 antibody (particularly an agonistic anti-CD40 antibody; e.g., CP-870,893 or Chi Lob 7/4). Further immunooncology therapeutics that can be used in combination with bendamustine (or a pharmaceutically acceptable salt or solvate thereof) are known in the art and are described, e.g., in: Kyi C et al., FEBS Lett, 2014, 588(2), 368-376; Intlekofer A M et al., J Leukoc Biol, 2013, 94(1), 25-39; Callahan M K et al., J Leukoc Biol, 2013, 94(1), 41-53; Ngiow S F et al., Cancer Res, 2011, 71(21), 6567-6571; or Blattman J N et al., Science, 2004, 305(5681), 200-205.

The composition (or pharmaceutical composition) according to the invention can also be administered in combination with physical therapy, such as radiotherapy. Radiotherapy may commence before, after, or simultaneously with administration of the composition of the invention. For example, radiotherapy may commence 1-10 minutes, 1-10 hours or 24-72 hours after administration of the composition according to the invention. Yet, these time frames are not to be construed as limiting. The subject is exposed to radiation, preferably gamma radiation, whereby the radiation may be provided in a single dose or in multiple doses that are administered over several hours, days and/or weeks. Gamma radiation may be delivered according to standard radiotherapeutic protocols using standard dosages and regimens.

The present invention thus relates to a composition (or a pharmaceutical composition) comprising bendamustine or a pharmaceutically acceptable salt or solvate thereof in combination with a modified cyclodextrin, as described and defined herein above, for use in the treatment of cancer, wherein the composition is to be administered in combination with one or more further anticancer agents and/or in combination with radiotherapy.

Preferably, the composition (or pharmaceutical composition) according to the invention is to be administered in combination with at least one further anticancer agent selected from etoposide, fludarabine, mitoxantrone, methotrexate, prednisone, vincristine, and an anti-CD20 monoclonal antibody. More preferably, the further anticancer agent is an anti-CD20 monoclonal antibody which is preferably selected from rituximab, ocrelizumab, ofatumumab, obinutuzumab (or afutuzumab), and ibritumomab tiuxetan (e.g., ⁹⁰Y-ibritumomab tiuxetan or ¹¹¹In-ibritumomab tiuxetan). Even more preferably, the further anticancer agent is rituximab or obinutuzumab, particularly rituximab. Accordingly, it is particularly preferred that the composition (or pharmaceutical composition) according to the invention is to be administered in combination with rituximab. The composition (or pharmaceutical composition) of the invention can also be administered in combination with rituximab and one or more further anticancer agents, including any of the above-mentioned exemplary anticancer agents (e.g., copanlisib). In a further embodiment, the composition (or pharmaceutical composition) according to the invention is to be administered in combination with obinutuzumab (particularly for the treatment of follicular non-Hodgkin lymphoma).

Yet, the oral formulations of bendamustine according to the invention can also be used in monotherapy, particularly in the monotherapeutic treatment of cancer (i.e., without administering any other anticancer agents until the treatment with the oral bendamustine formulation is terminated).

Moreover, the composition (or pharmaceutical composition) according to the present invention—either in combination with one or more further anticancer agents (including any of the exemplary anticancer agents described above, such as an anti-CD20 monoclonal antibody, particularly rituximab) or without any further anticancer agents—can also be administered in combination with an antiemetic agent. The antiemetic agent may, for example, be selected from alosetron, azasetron, bemesetron, cilansetron, clozapine, dazopride, dolasetron, granisetron, lerisetron, metoclopramide, mianserin, mirtazapine, olanzapine, ondansetron, palonosetron (e.g., palonosetron alone, or palonosetron in combination with netupitant), quetiapine, ramosetron, ricasetron, tropisetron, zatosetron, clozapine, cyproheptadine, hydroxyzine, olanzapine, risperidone, ziprasidone, dronabinol, nabilone, tetrahydrocannabinol, alizapride, bromopride, chlorpromazine, clebopride, domperidone, haloperidol, hydroxyzine, itopride, metoclopramide, metopimazine, prochlorperazine, thiethylperazine, trimethobenzamide, cyclizine, dimenhydrinate, diphenhydramine, hydroxyzine, meclizine, promethazine, atropine, diphenhydramine, hyoscyamine, scopolamine, aprepitant, casopitant, ezlopitant, fosaprepitant, maropitant, netupitant, rolapitant, vestipitant, cerium oxalate, dexamethasone, lorazepam, midazolam, propofol, or any combination thereof. Preferably, the antiemetic agent is a 5-HT₃ antagonist (or a “setron”), such as, e.g., alosetron, azasetron, bemesetron, cilansetron, clozapine, dazopride, dolasetron, granisetron, lerisetron, metoclopramide, mianserin, mirtazapine, olanzapine, ondansetron, palonosetron (optionally in combination with netupitant), quetiapine, ramosetron, ricasetron, tropisetron, or zatosetron. A particularly preferred antiemetic agent is palonosetron.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The terms “treatment”, “treating” and the like are used herein to refer to the obtainment of a desired pharmacological and/or physiological effect. The effect may be prophylactic in terms of completely or partially preventing a disease or symptom thereof and/or may be therapeutic in terms of partially or completely curing or halting a disease or symptom thereof and/or an adverse effect attributed to the disease. The term “treatment” as used herein covers any treatment of a disease in a patient and includes: (a) preventing a disease in a patient which may be predisposed/at risk of developing the disease; (b) inhibiting the disease, i.e. arresting its development; or (c) relieving the disease, i.e. causing regression of the disease. As used herein, the term “treating” a disease or “treatment” of a disease refers particularly to a slowing or a reversal of the progression of the disease. Treating a disease also includes treating a symptom and/or reducing the symptoms of the disease.

The term “hydrocarbon group” refers to a group consisting of carbon atoms and hydrogen atoms.

The term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₄ alkyl” denotes an alkyl group having 1 to 4 carbon atoms. Exemplary alkyl groups are methyl, ethyl, propyl (e.g., n-propyl or isopropyl), or butyl (e.g., n-butyl, isobutyl, sec-butyl, or tert-butyl).

As used herein, unless explicitly indicated otherwise or contradicted by context, the terms “a”, “an” and “the” are used interchangeably with “one or more” and “at least one”. Thus, for example, a composition comprising “an” excipient can be interpreted as referring to a composition comprising “one or more” excipients.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, if a component of a composition is indicated to be “optional”, the invention specifically relates to both possibilities, i.e., that the corresponding component is present (contained in the composition) or that the corresponding component is absent from the composition.

It is to be understood that the present invention specifically relates to each and every combination of features and embodiments described herein, including any combination of general and/or preferred features/embodiments.

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present invention relates.

Furthermore, provided herein is also a composition for use as a medicament (or, accordingly, a pharmaceutical composition for use in therapy), wherein the composition comprises bendamustine or a pharmaceutically acceptable salt or solvate thereof (e.g., bendamustine hydrochloride, particularly bendamustine hydrochloride monohydrate) in combination with sulfobutyl ether-β-cyclodextrin (SBE-β-CD; e.g., Captisol®), and wherein the composition is to be administered orally. The corresponding composition can be used for the same therapeutic applications (including, e.g., the treatment of cancer) as described herein in connection with the composition according to the present invention. The description of the general and preferred features of the composition of the present invention likewise applies to the composition provided in this paragraph, except that the latter contains SBE-β-CD in place of the modified cyclodextrin.

The present invention particularly relates to the following items:
1. A composition for use as a medicament, wherein the composition comprises bendamustine or a pharmaceutically acceptable salt or solvate thereof in combination with a modified cyclodextrin, wherein the composition is to be administered orally, and wherein said modified cyclodextrin is selected from α-cyclodextrin, β-cyclodextrin and γ-cyclodextrin, wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.
2. An oral pharmaceutical composition comprising bendamustine or a pharmaceutically acceptable salt or solvate thereof in combination with a modified cyclodextrin, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.
3. Use of bendamustine or a pharmaceutically acceptable salt or solvate thereof in combination with a modified cyclodextrin for the preparation of a medicament for oral administration, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.
4. A method of treating a disease or disorder in a subject in need thereof, the method comprising orally administering a pharmaceutical composition comprising bendamustine or a pharmaceutically acceptable salt or solvate thereof in combination with a modified cyclodextrin to the subject, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.
5. A method of delivering bendamustine or a pharmaceutically acceptable salt or solvate thereof to a subject in need thereof, the method comprising orally administering a pharmaceutical composition comprising bendamustine or a pharmaceutically acceptable salt or solvate thereof in combination with a modified cyclodextrin to the subject, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.
6. The composition for use according to item 1 or the oral pharmaceutical composition of item 2 or the use of item 3 or the method of item 4 or 5, wherein the composition comprises bendamustin hydrochloride.
7. The composition for use according to item 1 or the oral pharmaceutical composition of item 2 or the use of item 3 or the method of item 4 or 5, wherein the composition comprises bendamustin hydrochloride monohydrate.
8. The composition for use according to any one of items 1, 6 or 7 or the oral pharmaceutical composition of any one of items 2, 6 or 7 or the use of any one of items 3, 6 or 7 or the method of any one of items 4 to 7, wherein the modified cyclodextrin is β-cyclodextrin which is substituted with methyl, hydroxyethyl, hydroxypropyl, dihydroxypropyl, hydroxybutyl, acetyl, or any combination thereof.
9. The composition for use according to any one of items 1 or 6 to 8 or the oral pharmaceutical composition of any one of items 2 or 6 to 8 or the use of any one of items 3 or 6 to 8 or the method of any one of items 4 to 8, wherein the modified cyclodextrin is selected from methyl-β-cyclodextrin, hydroxypropyl-β-cyclodextrin, hydroxyethyl-β-cyclodextrin, dihydroxypropyl-β-cyclodextrin, hydroxybutyl-β-cyclodextrin, acetyl-β-cyclodextrin, and a β-cyclodextrin substituted with at least two different groups selected from methyl, hydroxyethyl, hydroxypropyl, dihydroxypropyl, hydroxybutyl, and acetyl.
10. The composition for use according to any one of items 1 or 6 to 9 or the oral pharmaceutical composition of any one of items 2 or 6 to 9 or the use of any one of items 3 or 6 to 9 or the method of any one of items 4 to 9, wherein the modified cyclodextrin is methyl-β-cyclodextrin.
11. The composition for use according to any one of items 1 or 6 to 9 or the oral pharmaceutical composition of any one of items 2 or 6 to 9 or the use of any one of items 3 or 6 to 9 or the method of any one of items 4 to 9, wherein the modified β-cyclodextrin is hydroxypropyl-β-cyclodextrin.
12. The composition for use according to any one of items 1 or 6 to 11 or the oral pharmaceutical composition of any one of items 2 or 6 to 11 or the use of any one of items 3 or 6 to 11 or the method of any one of items 4 to 11, wherein the composition comprises an inclusion complex of said modified cyclodextrin and said bendamustine or the pharmaceutically acceptable salt or solvate thereof.
13. The composition for use according to item 12 or the oral pharmaceutical composition of item 12 or the use of item 12 or the method of item 12, wherein the inclusion complex is obtainable by kneading, physically mixing, co-evaporating, freeze-drying, or spray-drying said modified cyclodextrin and said bendamustine or the pharmaceutically acceptable salt or solvate thereof.
14. The composition for use according to any one of items 1 or 6 to 13 or the oral pharmaceutical composition of any one of items 2 or 6 to 13 or the use of any one of items 3 or 6 to 13 or the method of any one of items 4 to 13, wherein the composition comprises said bendamustine or the pharmaceutically acceptable salt or solvate thereof and said modified cyclodextrin at a molar ratio of about 1:10 to about 1:0.5.
15. The composition for use according to any one of items 1 or 6 to 14 or the oral pharmaceutical composition of any one of items 2 or 6 to 14 or the use of any one of items 3 or 6 to 14 or the method of any one of items 4 to 14, wherein said composition is provided in the form of a solid oral dosage form, preferably in the form of a pill, a tablet, a mini-tablet, a capsule, a lozenge, a troche, a pellet, a powder, granules, or a film.
16. The composition for use according to item 15 or the oral pharmaceutical composition of item 15 or the use of item 15 or the method of item 15, wherein the solid oral dosage form has an enteric coating.
17. The composition for use according to item 16 or the oral pharmaceutical composition of item 16 or the use of item 16 or the method of item 16, wherein the enteric coating is made from a material selected from methyl acrylate-methacrylic acid copolymer, ethyl acrylate-methylacrylic acid copolymer, methyl methacrylate-methacrylic acid copolymer, cellulose acetate phthalate, cellulose acetate succinate, hydroxypropyl methyl cellulose, hydroxypropyl methyl cellulose phthalate, hydroxypropyl methyl cellulose acetate succinate, polyvinyl acetate phthalate, shellac, cellulose acetate trimellitate, carboxymethyl cellulose, sodium alginate, zein, amylose, starch, and dextrins.
18. A composition as defined in any one of items 1, 2 or 6 to 17 for use in the treatment of cancer, wherein the composition is to be administered orally.
19. The use of any one of items 3 or 6 to 17, wherein the medicament is for the treatment of cancer.
20. The method of any one of items 4 or 6 to 17, wherein the disease or disorder to be treated is cancer.
21. The composition for use according to item 18 or the use of item 19 or the method of item 20, wherein said cancer is a hematological cancer.
22. The composition for use according to item 21 or the use of item 21 or the method of item 21, wherein said cancer is a relapsed or refractory hematological cancer.
23. The composition for use according to item 21 or the use of item 21 or the method of item 21, wherein said cancer is a rituximab-refractory hematological cancer.
24. The composition for use according to any one of items 21 to 23 or the use of any one of items 21 to 23 or the method of any one of items 21 to 23, wherein said hematological cancer is selected from lymphoma, Hodgkin lymphoma, nodular sclerosing Hodgkin lymphoma, mixed-cellularity Hodgkin lymphoma, lymphocyte-rich Hodgkin lymphoma, lymphocyte-depleted Hodgkin lymphoma, nodular lymphocyte predominant Hodgkin lymphoma, non-Hodgkin lymphoma, follicular non-Hodgkin lymphoma, diffuse non-Hodgkin lymphoma, diffuse large B-cell lymphoma, Burkitt's lymphoma, mantle cell lymphoma, peripheral T-cell lymphoma, cutaneous T-cell lymphoma, mycosis fungoides, Sezary's disease, T-zone lymphoma, lymphoepithelioid lymphoma, Lennert's lymphoma, lymphosarcoma, a malignant immunoproliferative disease, Waldenström's macroglobulinemia, alpha heavy chain disease, gamma heavy chain disease, Franklin's disease, an immunoproliferative small intestinal disease, Mediterranean lymphoma, multiple myeloma, Kahler's disease, myelomatosis, leukemia, plasma cell leukemia, lymphoid leukemia, acute lymphoblastic leukemia, chronic lymphocytic leukemia, subacute lymphocytic leukemia, prolymphocytic leukemia, hairy-cell leukemia, leukemic reticuloendotheliosis, adult T-cell leukemia, myeloid leukemia, acute myeloid leukemia, chronic myeloid leukemia, subacute myeloid leukemia, myeloid sarcoma, chloroma, granulocytic sarcoma, acute promyelocytic leukemia, acute myelomonocytic leukemia, a chronic BCR-ABL negative myeloproliferative disorder, polycythaemia vera, essential thrombocythemia, idiopathic myelofibrosis, monocytic leukemia, acute erythraemia, erythroleukemia, acute erythraemic myelosis, Di Guglielmo's disease, chronic erythraemia, Heilmeyer-Schoner disease, acute megakaryoblastic leukemia, mast cell leukemia, acute panmyelosis, acute myelofibrosis, and Letterer-Siwe disease.
25. The composition for use according to any one of items 21 to 23 or the use of any one of items 21 to 23 or the method of any one of items 21 to 23, wherein said hematological cancer is selected from chronic lymphocytic leukemia, Hodgkin lymphoma, non-Hodgkin lymphoma, follicular non-Hodgkin lymphoma, indolent B cell non-Hodgkin lymphoma, mantle cell lymphoma, Waldenström's macroglobulinemia, and multiple myeloma.
26. The composition for use according to item 18 or the use of item 19 or the method of item 20, wherein said cancer is a solid cancer.
27. The composition for use according to item 18 or 26 or the use of item 19 or 26 or the method of item 20 or 26, wherein said cancer is selected from breast cancer, lung cancer, ovarian cancer, colorectal cancer, colon cancer, pancreatic cancer, bladder cancer, prostate cancer, head and/or neck cancer, and soft-tissue sarcoma.
28. A composition as defined in any one of items 1, 2 or 6 to 17 for use in the treatment of an autoimmune disease/disorder, rheumatoid arthritis, multiple sclerosis, lupus erythematosus, or a neurodegenerative disease/disorder, or for the use in immunomodulatory therapy, wherein the composition is to be administered orally.
29. The use of any one of items 3 or 6 to 17, wherein the medicament is for the treatment of an autoimmune disease/disorder, rheumatoid arthritis, multiple sclerosis, lupus erythematosus or a neurodegenerative disease/disorder, or the medicament is for immunomodulatory therapy.
30. The method of any one of items 4 or 6 to 17, wherein the disease or disorder to be treated is an autoimmune disease/disorder, rheumatoid arthritis, multiple sclerosis, lupus erythematosus, or a neurodegenerative disease/disorder.
31. The composition for use according to any one of items 18 or 21 to 28 or the use of any one of items 19, 21 to 27 or 29 or the method of any one of items 20 to 27 or 30, wherein the composition or medicament is to be administered in monotherapy.
32. The composition for use according to any one of items 18 or 21 to 27 or the use of any one of items 19 or 21 to 27 or the method of any one of items 20 to 27, wherein the composition or medicament is to be administered in combination with a further anticancer agent and/or radiotherapy.
33. The composition for use according to item 32 or the use of item 32 or the method of item 32, wherein said further anticancer agent is selected from etoposide, fludarabine, mitoxantrone, methotrexate, prednisone, vincristine, and an anti-CD20 monoclonal antibody.
34. The composition for use according to item 32 or the use of item 32 or the method of item 32, wherein said further anticancer agent is an anti-CD20 monoclonal antibody which is preferably selected from rituximab, ocrelizumab, ofatumumab, obinutuzumab, and ibritumomab tiuxetan.
35. The composition for use according to any one of items 18 or 21 to 28 or the use of any one of items 19, 21 to 27 or 29 or the method of any one of items 20 to 27 or 30, wherein the composition or medicament is to be administered in combination with rituximab.
36. The composition for use according to any one of items 18 or 21 to 28 or the use of any one of items 19, 21 to 27 or 29 or the method of any one of items 20 to 27 or 30, wherein the composition or medicament is to be administered in combination with obinutuzumab.
37. The composition for use according to any one of items 1, 6 to 18, 21 to 28 or 32 to 36 or the use of any one of items 3, 6 to 17, 19, 21 to 27, 29 or 32 to 36 or the method of any one of items 4 to 17, 20 to 27, 30 or 32 to 36, wherein the composition or medicament is to be administered in combination with an antiemetic agent.
38. The composition for use according to item 37 or the use of item 37 or the method of item 37, wherein said antiemetic agent is selected from alosetron, azasetron, bemesetron, cilansetron, clozapine, dazopride, dolasetron, granisetron, lerisetron, metoclopramide, mianserin, mirtazapine, olanzapine, ondansetron, palonosetron, quetiapine, ramosetron, ricasetron, tropisetron, zatosetron, clozapine, cyproheptadine, hydroxyzine, olanzapine, risperidone, ziprasidone, dronabinol, nabilone, tetrahydrocannabinol, alizapride, bromopride, chlorpromazine, clebopride, domperidone, haloperidol, hydroxyzine, itopride, metoclopramide, metopimazine, prochlorperazine, thiethylperazine, trimethobenzamide, cyclizine, dimenhydrinate, diphenhydramine, hydroxyzine, meclizine, promethazine, atropine, diphenhydramine, hyoscyamine, scopolamine, aprepitant, casopitant, ezlopitant, fosaprepitant, maropitant, netupitant, rolapitant, vestipitant, cerium oxalate, dexamethasone, lorazepam, midazolam, propofol, and a combination thereof.
39. The composition for use according to any one of items 1, 6 to 18, 21 to 28 or 31 to 38 or the use of any one of items 3, 6 to 17, 19, 21 to 27, 29 or 31 to 38 or the method of any one of items 4 to 17, 20 to 27, 30 to 38, wherein the composition or medicament is to be administered orally to a human subject.
40. The composition for use according to any one of items 1, 6 to 18, 21 to 28 or 31 to 39 or the use of any one of items 3, 6 to 17, 19, 21 to 27, 29 or 31 to 39 or the method of any one of items 4 to 17, 20 to 27, 30 to 39, wherein the composition or medicament is to be administered orally in a unit dose of about 10 mg to about 1 g, preferably in a unit dose of about 20 mg to about 800 mg, more preferably in a unit dose of about 30 mg to about 600 mg, even more preferably in a unit dose of about 50 mg to about 500 mg.

41. The composition for use according to item 40 or the use of item 40 or the method of item 40, wherein the unit dose is to be administered one to five times every three or four weeks.
42. The composition for use according to any one of items 1, 6 to 18, 21 to 28 or 31 to 39 or the use of any one of items 3, 6 to 17, 19, 21 to 27, 29 or 31 to 39 or the method of any one of items 4 to 17, 20 to 27, 30 to 39, wherein the composition or medicament is to be administered orally in a unit dose of about 50 mg to about 500 mg, and wherein the unit dose is to be administered on days 1 and 2 every 21 days.
43. The composition for use according to any one of items 1, 6 to 18, 21 to 28 or 31 to 39 or the use of any one of items 3, 6 to 17, 19, 21 to 27, 29 or 31 to 39 or the method of any one of items 4 to 17, 20 to 27, 30 to 39, wherein the composition or medicament is to be administered orally in a unit dose of about 50 mg to about 500 mg, and wherein the unit dose is to be administered on days 1 to 5 every 21 days.
44. The composition for use according to any one of items 1, 6 to 18, 21 to 28 or 31 to 39 or the use of any one of items 3, 6 to 17, 19, 21 to 27, 29 or 31 to 39 or the method of any one of items 4 to 17, 20 to 27, 30 to 39, wherein the composition or medicament is to be administered orally in a unit dose of about 50 mg to about 500 mg, and wherein the unit dose is to be administered on days 1 and 2 every 28 days.
45. The composition for use according to any one of items 1, 6 to 18, 21 to 28 or 31 to 39 or the use of any one of items 3, 6 to 17, 19, 21 to 27, 29 or 31 to 39 or the method of any one of items 4 to 17, 20 to 27, 30 to 39, wherein the composition or medicament is to be administered orally in a unit dose of about 50 mg to about 500 mg, and wherein the unit dose is to be administered on days 1 and 15 every 28 days.
46. The composition for use according to any one of items 1, 6 to 18, 21 to 28 or 31 to 39 or the use of any one of items 3, 6 to 17, 19, 21 to 27, 29 or 31 to 39 or the method of any one of items 4 to 17, 20 to 27, 30 to 39, wherein the composition or medicament is to be administered orally in a unit dose of about 50 mg to about 500 mg, and wherein the unit dose is to be administered on days 1 to 5 every 28 days.
47. The composition for use according to any one of items 40 to 46 or the use of any one of items 40 to 46 or the method of any one of items 40 to 46, wherein the unit dose is selected from about 50 mg, about 75 mg, about 100 mg, about 150 mg, about 180 mg, about 200 mg, about 250 mg, about 300 mg, about 350 mg, about 400 mg, about 450 mg, and about 500 mg.

The invention will now be described by reference to the following examples which are merely illustrative and are not to be construed as a limitation of the scope of the present invention.

### EXAMPLES

In-Vivo PK Studies of Different Formulations

As bioanalytical method a fast and reliable method was used to determine bendamustine and its main metabolite γ-hydroxybendamustine (M3 metabolite) simultaneously in plasma of different species (Srinivas N R et al., Drug Res (Stuttg), 2016, 66(7), 351-356; Chandrashekar D V et al., Drug Res (Stuttg), 2017, doi: 10.1055/s-0043-108124).

Absolute bioavailability refers to the bioavailability of drug when administered via a non-intravenous dosage form (i.e. oral) compared with the bioavailability of the same drug administered intravenously. It is calculated by employing the following formula:

$$Absolute oral bioavailability F = \frac{A U {C}_{P O} \times D o s {e}_{IV}}{A U {C}_{IV} \times D o s {e}_{P O}}$$

The pharmacokinetic parameters determined in all PK studies are as defined as follows:

| Parameter | Unit | Description |
|---|---|---|
| t_{1/2, ß} | h | Terminal elimination half-life is the time |
| | | the drug concentration needs to decrease |
| | | by 50%. |
| AUC | ng · h/mL | Area under the plasma concentration - |
| | | time curve. |
| Cₘₐₓ | ng/mL | Peak plasma concentration. |
| Cl | mL/min/kg | It is the measurement of the volume of |
| | | plasma/blood that is completely cleared |
| | | off of the compound per unit time. |
| MRT | h | Mean residence time is time that the |
| | | compound, on average, resides in the |
| | | body. |
| Vss | L/kg | A measure of the theoretical volume that |
| | | a compound distributes at steady state. |
| Vz | L/kg | Refers to the amount of drug in body/ |
| | | concentration in plasma. |
| Bioavailability | F | The fraction of the administered dose that |
| | | reaches the systemic circulation. |

### Example 1

### Oral Bioavailability of Bendamustine Using Captisol® in Sprague Dawley (SD) Rats

Captisol is a polyanionic betα-cyclodextrin derivative with a sodium sulfonate salt separated from the lipophilic cavity by a butyl ether spacer group, or sulfobutylether (SBE) (www.captisol.com).

The preparation of the formulation was performed by mixing bendamustine hydrochloride and Captisol® as a mixture in a 1:2 molar ratio. The formulation was freshly prepared on the day of dosing by dissolving it in an adequate amount of Milli-Q water and administered within 5 min to the rats per required dosing volume. Approximately 0.2 mL of blood samples were collected from each animal at pre-determined time points post formulation administration under isoflurane anesthesia.

The results obtained are shown in the following Table 1:

TABLE 1
Pharmacokinetic parameters of bendamustine following oral
administration of Bendamustine-Captisol ® Formulation
at 9.0 mg/kg (equivalent to bendamustine base) to male SD rats.

| PK parameter | | Mean | S.D. | CV % |
|---|---|---|---|---|
| t_{1/2, ß} | h | 0.92 | 0.54 | 58.9 |
| AUC₀₋ₜ | ng · h/mL | 1591 | 819 | 51.5 |
| AUC_{0-∞} | ng · h/mL | 1596 | 820 | 51.4 |
| Cₘₐₓ | ng/mL | 2357 | 1084 | 46.0 |
| MRT | h | 0.84 | 0.11 | 13.4 |
| Tₘₐₓ | h | 0.44 | 0.24 | 54.7 |

| | | |
|---|---|---|
| Absolute Oral | using 30 min IV infusion data^{#} | 67% |
| bioavailability F (%) | using 60 min IV infusion data^{@} | 90% |

^{#}i.v. data derived from previous experiment
^{@}i.v. data derived from previous experiment

The ratio of the AUC_{0->∞} of the corresponding γ-hydroxybendamustine metabolite (M3) to bendamustine was determined to be 0.09.

### Example 2

### Oral Bioavailability of Eendamustin Using β-Cyclodextrin in SD Rats

The preparation of the formulation was performed by mixing bendamustine hydrochloride and β-cyclodextrin as a mixture in a 1:4 molar ratio. The formulation was freshly prepared on the day of dosing by dissolving it in an adequate amount of Milli-Q water and administered within 5 min to the rats per required dosing volume. Approximately 0.2 mL of blood samples were collected from each animal at pre-determined time points post formulation administration under isoflurane anesthesia.

The results obtained are shown in the following Table 2:

TABLE 2
(Group-2):
Pharmacokinetic parameters of bendamustine following oral
administration of Bendamustine-β-Cyclodextrin at 9.0 mg/kg
(equiv. to bendamustine base) to male SD rats.

| | | | |
|---|---|---|---|
| PK parameter | Mean | S.D. | CV % |

| | | | | |
|---|---|---|---|---|
| t_{1/2, ß} | h | 0.94 | 0.39 | 42 |
| AUC₀₋ₜ | ng · h/mL | 1420 | 598 | 42.1 |
| AUC_{0-∞} | ng · h/mL | 1426 | 598 | 42.0 |
| Cₘₐₓ | ng/mL | 2533 | 680 | 26.9 |
| MRT | h | 0.65 | 0.08 | 12.0 |
| Tₘₐₓ | h | 0.25 | 0.00 | 0.00 |

| | | |
|---|---|---|
| Absolute Oral | using 30 min IV infusion data^{#} | 60% |
| bioavailability F (%) | using 60 min IV infusion data^{@} | 80% |

^{#}IV data derived from previous experiment
^{@}IV data derived from previous experiment

The ratio of the AUC_{0->∞} of the corresponding γ-hydroxybendamustine metabolite (M3) to bendamustine was determined to be 0.05.

### Example 3

### Oral Bioavailability of Bendamustin Using α-Cyclodextrin in SD Rats

The preparation of the formulation was performed by mixing bendamustine hydrochloride and α-cyclodextrin as a mixture in a 1:4.5 molar ratio. The formulation was freshly prepared on the day of dosing by dissolving it in an adequate amount of Milli-Q water and administered within 5 min to the rats per required dosing volume. Approximately 0.2 mL of blood samples were collected from each animal at pre-determined time points post formulation administration under isoflurane anesthesia.

The results obtained are shown in the following Table 3:

TABLE 3
Pharmacokinetic parameters of bendamustine following oral
administration of Bendamustine-α-Cyclodextrin at 9.0
mg/kg (equivalent to bendamustine base) to male SD rats.

| | | | |
|---|---|---|---|
| PK parameter | Mean | S.D. | CV % |

| | | | | |
|---|---|---|---|---|
| t_{1/2, ß} | h | 0.95 | 0.58 | 60.9 |
| AUC₀₋ₜ | ng · h/mL | 761 | 198 | 26.0 |
| AUC_{0-∞} | ng · h/mL | 781 | 207 | 26.5 |
| Cₘₐₓ | ng/mL | 1188 | 474 | 39.9 |
| MRT | h | 0.98 | 0.50 | 51.0 |
| Tₘₐₓ | h | 0.25 | 0.00 | 0.00 |

| | | |
|---|---|---|
| Absolute Oral | using 30 min IV infusion data^{#} | 33% |
| bioavailability F (%) | using 60 min IV infusion data^{@} | 44% |

^{#}IV data derived from previous experiment
^{@}IV data derived from previous experiment

The ratio of the AUC_{0->∞} of the corresponding γ-hydroxybendamustine metabolite (M3) to bendamustine was determined to be 0.16.

The ratio of the AUC_{0->∞} of the corresponding γ-hydroxybendamustine metabolite (M3) to bendamustine was determined to be 0.07 and 0.05, resp. for 30 min and 60 min i.v. administration, resp.

### Example 4

### Oral Bioavailability of Bendamustine Using Methyl-β-Cyclodextrin in SD Rats

The preparation of the formulation was performed by mixing bendamustine hydrochloride and methyl-β-cyclodextrin as a mixture in a 1:1 molar ratio. The formulation was freshly prepared on the day of dosing by dissolving it in an adequate amount of Milli-Q water and administered within 5 min to the rats per required dosing volume. Approximately 0.2 mL of blood samples were collected from each animal at pre-determined time points post formulation administration under isoflurane anesthesia.

The relative oral bioavailability was determined with the following formula:

$$Relative oral bioavailability ( F ) = \frac{A U {C}_{POreference} \times D o s {e}_{POstandard}}{A U {C}_{{e}_{POstandard}} \times D o s {e}_{POreference}}$$

The results obtained are shown in the following Table 4.

TABLE 4
Pharmacokinetic parameters of bendamustine following
oral administration of Bendamustine-Methyl-β-Cyclodextrin
formulation at 15 mg/kg to male SD rats.

| PK parameter | | Mean | S.D. | CV % |
|---|---|---|---|---|
| t_{1/2} | h | 0.50 | 0.49 | 98.2 |
| AUC₀₋ₜ | ng · h/mL | 4201 | 1792 | 42.7 |
| AUC_{0-∞} | ng · h/mL | 4189 | 1800 | 43.0 |
| Cₘₐₓ | ng/mL | 7312 | 4408 | 60.3 |
| Cl/F | mL/min/Kg | 67.4 | 25.1 | 37.2 |
| MRT | h | 0.57 | 0.12 | 20.8 |
| Vz/F | L/Kg | 2.89 | 3.16 | 109.1 |

| | |
|---|---|
| oral bioavailability F* | 140% comp. 60 min IV infusion time |
| | 105% comp. 30 min IV infusion time |
| | 185% relative to oral bendamustine in |
| | water |

*calculated as the quotient of AUC_{0-∞} (oral) and AUC_{0-∞} (iv; Group 1) with appropriate dose normalization times 100

The oral bioavailability of the M3 metabolite is 176% compared to 60 min IV infusion time and 78% compared to 30 min IV infusion time.

The ratio of the AUC_{0->∞} of the corresponding γ-hydroxybendamustine metabolite (M3) to bendamustine was determined to be 0.05 which corresponds almost equally to the IV infusion time.

### Example 5

### Oral Bioavailability of Bendamustin Using a β-Cyclodextrin Formulation in Balb/c Mice

The preparation of the formulation was performed by mixing bendamustine hydrochloride and β-cyclodextrin as a mixture in a 1:6 molar ratio. The formulation was freshly prepared on the day of dosing by dissolving it in an adequate amount of Milli-Q water and administered within 5 min to the mice per required dosing volume. Sparse sampling (n=3 at each time point) was done and each animal was bled twice. Blood samples (˜200 μL) were collected in tubes containing Na₂.EDTA at the below mentioned time points post compound administration under isoflurane anesthesia.

TABLE 5
Pharmacokinetic parameters of bendamustine following
oral administration of Bendamustine-β-Cyclodextrin
formulation (equivalent to 9 mg/kg of bendamustine
base) to male Balb/c mice.

| Parameter | Unit | Bendamustine |
|---|---|---|
| t_{1/2, ß} | h | 0.81 |
| tₘₐₓ | h | 0.25 |
| Cₘₐₓ | ng/mL | 5329 |
| Tₗₐₛₜ | h | 8.00 |
| AUC₀₋ₜ | h*ng/mL | 4882 |
| AUC_{0-∞} | h*ng/mL | 4888 |

| | |
|---|---|
| Oral bioavailability (F %) | 93 |

### Example 6

### Oral Bioavailability of Bendamustine Using a β-Cyclodextrin and SNAC Formulation in SD Rats

The preparation of the formulation was performed by mixing bendamustine hydrochloride and β-cyclodextrin and sodium N-[8-(2-hydroxybenzoyl)amino]caprylate (SNAC)* as a mixture in a 1:6:22 molar ratio. The formulation was freshly prepared on the day of dosing by dissolving it in an adequate amount of Milli-Q water and administered within 5 min to the rats per required dosing volume. Approximately 0.2 mL of blood samples were collected from each animal at pre-determined time points post formulation administration under isoflurane anesthesia. *SNAC is a enhancer of oral bioavailability (Castelli M C et al., The FASEB Journal, 2008, 22(2) Suppl., 795).

The results obtained are shown in the following Table 6:

TABLE 6
Pharmacokinetic parameters of bendamustine following oral
administration of Bendamustine-β-Cyclodextrine-SNAC at
9 mg/kg (equivalent to bendamustine base) to male SD rats.

| PK parameter | | Mean | S.D. | CV % |
|---|---|---|---|---|
| t_{1/2, ß} | h | 0.70 | 0.08 | 10.9 |
| Tₘₐₓ | h | 0.25 | 0.00 | 0.00 |
| AUC₀₋ₜ | ng · h/mL | 1392 | 209 | 15.0 |
| AUC_{0-∞} | ng · h/mL | 1395 | 209 | 15.0 |
| Cₘₐₓ | ng/mL | 1665 | 199 | 12.0 |
| Tₗₐₛₜ | h | 6.00 | 0.00 | 0.0 |

| Oral bioavailability F (%): | 79% comp. 60 min IV infusion time |
|---|---|
| | 79% comp. 30 min IV infusion time |
| | 103% relative to oral bendamustine |
| | in water |

### Example 7

### Comparison of Different Formulations of Bendamustine and its Metabolite M3 in PK Studies in Beagle Dogs

The oral bioavailability and the PK profile of formulations of bendamustine with 2-hydroxypropyl-β-cyclodextrin and β-cyclodextrin were compared in beagle dogs.

Capsules with an amount of bendamustine hydrochloride for each of the two formulations to reach a dose of 12 mg/kg were administered to beagle dogs using a cross over design and compared to 60 min IV infusion administration. Capsule formulations were stored between 2-8° C. The respective IV formulation was prepared fresh immediately prior to dosing.

The results obtained are shown in the following Table 7:

TABLE 7
Comparative table of PK data of two
bendamustine-formulations in dogs.

| | | Oral | IV |
|---|---|---|---|
| Formulation type | PK Parameters | (capsule) | (60 min) |
| Bendamustine- | Cₘₐₓ [ng/mL] | 7802 | 4304 |
| 2HP-β-CD | AUC_{0 >∞} [ng · h/mL] | 3785 | 3805 |
| | F [%] | 99 | |
| Bendamustine-β-CD | Cₘₐₓ [ng/mL] | 2564 | |
| | AUC_{0 >∞} [ng · h/mL] | 1928 | |
| | F [%] | 51 | |

The ratio of the AUC_{0->∞} of the corresponding γ-hydroxybendamustine metabolite (M3) to bendamustine was determined to be 0.03 for the 2HP-β-CD formulation which corresponds almost equally to the IV infusion time for 60 min with a ratio of 0.08.

The ratio of the β-CD formulation was determined to be 0.26.

The close ratio between the AUCs of M3 and bendamustine for the 2HP-β-CD formulation and the intravenous application indicate that the metabolism of bendamustine is the same in both administrations.

Stability of Bendamustine Formulations in Water

### Example 8

### Comparison of Stability of Bendamustine Formulations with 2-Hydroxypropyl-β-Cyclodextrin and Methyl-β-Cyclodextrin Obtained by Kneading in Water/Ethanol

2-Hydroxypropyl-β-cyclodextrin formulation: 1.25 mmol bendamustine hydrochloride was mixed with 1.25 mmol 2HP-β-CD (MW=1540), wetted with an equimolar mixture of EtOH/H₂O and kneaded for ca. 20 min. Then the clear solution was evaporated at 40° C. bath temperature in vacuo, dried further in high vacuum and stored under vacuum in a desiccator for 5 days.

Methyl-β-cyclodextrin formulation: 1.25 mmol bendamustine hydrochloride was mixed with 1.25 mmol Me-β-CD (MW=1310), wetted with an equimolar mixture of EtOH/H₂O and kneaded for ca. 20 min. Then the clear solution was evaporated at 40° C. bath temperature in vacuo, dried further in high vacuum and stored under vacuum in a desiccator for 5 days.

10 mg of each bendamustine formulation freshly prepared were measured with HPLC and the hydrolytic stability of bendamustine was determined over 3 hours.

After 180 min 89.40% of bendamustine were present with the methyl-β-cyclodextrin formulation and 88.94% of the 2-hydroxypropyl-β-cyclodextrin formulation. Over the time of 180 min, the methyl-β-cyclodextrin formulation and the 2-hydroxypropyl-β-cyclodextrin formulation were thus found to have an advantageous and almost equally potent stabilizing effect on bendamustine.

Efficacy Studies of Bendamustine Formulations

### Example 9

### B-Cell Lymphoma Xenograft Study with Me-β-CD and 2-HP-β-CD formulations of Bendamustine

To groups of eight male NOD/SCID mice each were injected Raji cells and when the tumor volume reached ˜150 mm³ the mice were each treated with vehicle, bendamustine intravenously, bendamustine in water orally, bendamustine-Me-β-CD formulation orally and bendamustine-2HP-β-CD formulation, respectively. The treatment for all groups were once/week for 2 weeks on day 1 and on day 8. Tumor volume and body weight were measured for four weeks. On day 30 the study was terminated.

The tumor volumes of both oral formulations, i.e. bendamustine-2HP-β-CD orally and bendamustine-Me-β-CD orally, were equal over the time of the study and clearly inhibited the tumor growth equally as compared with the bendamustine intravenous application. The inhibitory effect of bendamustine in water was less compared with the oral or intravenous applications (see FIG. 2).

Remarkably the body weight change was less in the oral bendamustine formulations compared to the intravenous applications (see FIG. 3), which indicates that the oral bendamustine-Me-β-CD and the oral bendamustine-2HP-β-CD formulations are better tolerated.

Comparative Studies of Bendamustine Formulations

### Example 10

### Comparison of Bendamustine Formulations with 2-Hydroxypropyl-β-Cyclodextrin or Methyl-β-Cyclodextrin Obtained by Kneading and by Physical Mixing, Respectively

In a PK study the differences in oral bioavailability (F) between randomized methyl-ß-cyclodextrin and 2-HP-ß-cyclodextrin prepared by kneading (see the procedure described in Example 8 above) and as a physical mixture, respectively, were assessed.

Results: With both cyclodextrins the oral bioavailability was lower with the kneading procedure than by using a physical mixture.

Methyl-ß-cyclodextrin—Bendamustine HCl ratio 1:1 (MIM): Kneading vs. Physical Mixture ratio 1:1 (M/M): Factor (ratio between kneading/physical mixture)=0.6

2-Hydroxypropyl-ß-cyclodextrin—Bendamustine HCl ratio 1:1 (M/M): Kneading vs. Physical Mixture ratio 1:1 (M/M): Factor (ratio between kneading/physical mixture)=0.8

### Example 11

### Comparison of Bendamustine Formulations with Polymerized Epichlorohydrin-ß-Cyclodextrin (Reference), 2-Hydroxypropyl-β-Cyclodextrin, and Methyl-β-Cyclodextrin

As comparison a bendamustine formulation with epichlorohydrin-ß-cyclodextrin polymerized ratio 1:1 (M/M) was tested in a PK study: absolute bioavailability is 85%; relative bioavailability is 214%, which is significantly lower than the value of 323% relative bioavailability stated in the publication from Gidwani B et al., Drug Dev Ind Pharm, 2015, 41(12), 1978-1988. The absolute oral bioavailability for the epichlorohydrin-ß-cyclodextrine formulation was not given in Gidwani, and the pharmacokinetic parameters such as Cₘₐₓ and AUC are significantly higher in the publication from Gidwani than experimentally measured in this Example, as detailed in the following.

Published PK values for bendamustin in water (10 mg/kg): Cₘₐₓ=12.6 μg/mL; AUC=13.2 μg·h/mL

Experimentally established PK values for bendamustin in water (15 mg/kg): Cₘ=2.6 μg/mL; AUC=1.35 μg·h/mL

Published PK values for epichlorohydrin-ß-CD formulation kneaded/ bendamustine (1:1 M/M) (10 mg/kg): Cₘₐₓ=32.13 μg/mL; AUC=42.64 μg·h/mL

Experimentally established PK values for epichlorohydrin-ß-CD formulation kneaded/bendamustine (1:1 M/M) (15 mg/kg): Cₘₐₓ=5.1 μg/mL; AUC=2.9 μg·h/mL

Additionally, the t_{1/2} was determined for the epichlorohydrin-ß-CD formulation as 25 min in contrast to the published one of 74 min. In strong contrast, the t_{1/2} of the physical 3:1 (M/M) formulation of 2-HP-ß-CD with bendamustine was determined to be 68 min.

The absolute bioavailability of the 2-hydroxypropyl-ß-cyclodextrine formulation of bendamustine HCl (3:1 M/M) was 58% and the relative bioavailability was determined to be 144%.

The results obtained are also shown in FIG. 4.

The pharmacokinetics of the formulations with randomized methyl-ß-CD (rMeCD) and 2-hydroxypropyl-ß-CD (2HPCD) show a favorable profile over the bendamustine given intravenously and even the polymerized epichlorohydrin-ß-CD (EpiCDp) regarding a longer exposure over time, clearly indicating a stabilization effect of bendamustine.

The ratio of the AUC_{0->∞} of the corresponding γ-hydroxybendamustine metabolite (M3) to bendamustine was determined to be 0.02 for the 2HP-ß-CD formulation, which corresponds almost equally to the IV infusion time for 30 min with a ratio of 0.01.

The ratio of the AUC_{0->∞} of the corresponding γ-hydroxybendamustine metabolite (M3) to bendamustine was determined to be 0.027 for the rMe-ß-CD formulation, which corresponds almost equally to the IV infusion time for 30 min with a ratio of 0.01.

### Example 12

### Comparison of Stability of Bendamustine Formulations

The stability of bendamustine formulations with 2-hydroxypropyl-ß-cyclodextrine obtained as a physical mixture with different molar ratios, such as e.g. 1:0.5; 1:0.7; 1:0.9; 1:1; 1:1.5; 1:2; 1:5; 1:7; 1:10 was compared. As an example one typical procedure is outlined here which is also true for other molar ratios determined:

0.5 mmol bendamustine HCl was mixed with 1.5 mmol 2-hydroxypropyl-ß-cyclodextrine physically by thoroughly shaking the flask for 3 hours. 10 mg of the bendamustine formulation were measured by HPLC every 30 min for 180 min total to determine the hydrolytic stability. After 180 min 97% of bendamustine HCl were present.

0.2 mmol bendamustine HCl was mixed with 0.2 mmol epichlorohydrin-ß-cyclodextrine (reference) and kneaded as described in Example 8 above. 10 mg of the bendamustine formulation were measured by HPLC every 30 min for 180 min total to determine the hydrolytic stability. After 180 min 94% of bendamustine HCl were present.

The hydrolytic stability of bendamustine formulations with randomized methyl-ß-cyclodextrine obtained as a physical mixture with different molar ratios was compared. As an example one typical procedure is outlined here which is also true for other molar ratios determined, such as e.g. 1:0.5; 1:0.7; 1:0.9; 1:1; 1:1.5; 1:2; 1:3; 1:5; 1:10:

0.5 mmol bendamustine HCl was mixed with 0.9 mmol randomized methyl-ß-cyclodextrine physically by thoroughly shaking the flask for 3 hours. 10 mg of the bendamustine formulation were measured by HPLC every 30 min for 180 min total to determine the hydrolytic stability. After 180 min 98% of bendamustine HCl were present.

The hydrolytic stability of bendamustine formulations with mixtures of randomized methyl-ß-cyclodextrine and 2-hydroxypropyl-ß-cyclodextrine obtained as a physical mixture with different molar ratios was compared. As an example one typical procedure is outlined here which is also true for other molar ratios determined, such as e.g. 1:0.5:0.5; 1:0.7:1.3; 1:0.9;1.1; 1:1:2; 1:1:3; 1:1:5; 1:2.6; 1:3:7:

0.5 mmol bendamustine HCl was mixed with 0.7 mmol randomized methyl-ß-cyclodextrine and 1.3 mmol 2-hydroxypropyl-ß-cyclodextrine physically by thoroughly shaking the flask for 3 hours. 10 mg of the bendamustine formulation were measured by HPLC every 30 min for 180 min total to determine the hydrolytic stability. After 180 min 98.5% of bendamustine HCl were present.

1. A composition for use as a medicament, wherein the composition comprises bendamustine or a pharmaceutically acceptable salt or solvate thereof in combination with a modified cyclodextrin, wherein the composition is to be administered orally, and wherein said modified cyclodextrin is selected from α-cyclodextrin, β-cyclodextrin and γ-cyclodextrin, wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

2. An oral pharmaceutical composition comprising bendamustine or a pharmaceutically acceptable salt or solvate thereof in combination with a modified cyclodextrin, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

3. The composition for use according to claim 1 or the oral pharmaceutical composition of claim 2, wherein the composition comprises bendamustin hydrochloride.

4. The composition for use according to claim 1 or the oral pharmaceutical composition of claim 2, wherein the composition comprises bendamustin hydrochloride monohydrate.

5. The composition for use according to any one of claim 1, 3 or 4 or the oral pharmaceutical composition of any one of claims 2 to 4, wherein the modified cyclodextrin is β-cyclodextrin which is substituted with methyl, hydroxyethyl, hydroxypropyl, dihydroxypropyl, hydroxybutyl, acetyl, or any combination thereof.

6. The composition for use according to any one of claims 1 or 3 to 5 or the oral pharmaceutical composition of any one of claims 2 to 5, wherein the modified cyclodextrin is selected from methyl-β-cyclodextrin, hydroxypropyl-β-cyclodextrin, hydroxyethyl-β-cyclodextrin, di hyd roxypropyl-β-cyclodextrin, hydroxybutyl-β-cyclodextrin, acetyl-β-cyclodextrin, and a β-cyclodextrin substituted with at least two different groups selected from methyl, hydroxyethyl, hydroxypropyl, dihydroxypropyl, hydroxybutyl, and acetyl.

7. The composition for use according to any one of claims 1 or 3 to 6 or the oral pharmaceutical composition of any one of claims 2 to 6, wherein the modified cyclodextrin is methyl-β-cyclodextrin.

8. The composition for use according to any one of claims 1 or 3 to 6 or the oral pharmaceutical composition of any one of claims 2 to 6, wherein the modified β-cyclodextrin is hydroxypropyl-β-cyclodextrin.

9. The composition for use according to any one of claims 1 or 3 to 8 or the oral pharmaceutical composition of any one of claims 2 to 8, wherein the composition comprises an inclusion complex of said modified cyclodextrin and said bendamustine or the pharmaceutically acceptable salt or solvate thereof, wherein the inclusion complex is obtainable by kneading, physically mixing, co-evaporating, freeze-drying, or spray-drying said modified cyclodextrin and said bendamustine or the pharmaceutically acceptable salt or solvate thereof.

10. The composition for use according to any one of claims 1 or 3 to 9 or the oral pharmaceutical composition of any one of claims 2 to 9, wherein the composition comprises said bendamustine or the pharmaceutically acceptable salt or solvate thereof and said modified cyclodextrin at a molar ratio of about 1:10 to about 1:0.5.

11. The composition for use according to any one of claims 1 or 3 to 10 or the oral pharmaceutical composition of any one of claims 2 to 10, wherein said composition is provided in the form of a solid oral dosage form, preferably in the form of a pill, a tablet, a mini-tablet, a capsule, a lozenge, a troche, a pellet, a powder, granules, or a film; and wherein the solid oral dosage form optionally has an enteric coating, wherein the enteric coating is preferably made from a material selected from methyl acrylate-methacrylic acid copolymer, ethyl acrylate-methylacrylic acid copolymer, methyl methacrylate-methacrylic acid copolymer, cellulose acetate phthalate, cellulose acetate succinate, hydroxypropyl methyl cellulose, hydroxypropyl methyl cellulose phthalate, hydroxypropyl methyl cellulose acetate succinate, polyvinyl acetate phthalate, shellac, cellulose acetate trimellitate, carboxymethyl cellulose, sodium alginate, zein, amylose, starch, and dextrins.

12. A composition as defined in any one of claims 1 to 11 for use in the treatment of cancer, wherein the composition is to be administered orally.

13. The composition for use according to claim 12, wherein said cancer is a hematological cancer which is preferably selected from lymphoma, Hodgkin lymphoma, nodular sclerosing Hodgkin lymphoma, mixed-cellularity Hodgkin lymphoma, lymphocyte-rich Hodgkin lymphoma, lymphocyte-depleted Hodgkin lymphoma, nodular lymphocyte predominant Hodgkin lymphoma, non-Hodgkin lymphoma, follicular non-Hodgkin lymphoma, diffuse non-Hodgkin lymphoma, diffuse large B-cell lymphoma, Burkitt's lymphoma, mantle cell lymphoma, peripheral T-cell lymphoma, cutaneous T-cell lymphoma, mycosis fungoides, Sézary's disease, T-zone lymphoma, lymphoepithelioid lymphoma, Lennert's lymphoma, lymphosarcoma, a malignant immunoproliferative disease, Waldenström's macroglobulinemia, alpha heavy chain disease, gamma heavy chain disease, Franklin's disease, an immunoproliferative small intestinal disease, Mediterranean lymphoma, multiple myeloma, Kahler's disease, myelomatosis, leukemia, plasma cell leukemia, lymphoid leukemia, acute lymphoblastic leukemia, chronic lymphocytic leukemia, subacute lymphocytic leukemia, prolymphocytic leukemia, hairy-cell leukemia, leukemic reticuloendotheliosis, adult T-cell leukemia, myeloid leukemia, acute myeloid leukemia, chronic myeloid leukemia, subacute myeloid leukemia, myeloid sarcoma, chloroma, granulocytic sarcoma, acute promyelocytic leukemia, acute myelomonocytic leukemia, a chronic BCR-ABL negative myeloproliferative disorder, polycythaemia vera, essential thrombocythemia, idiopathic myelofibrosis, monocytic leukemia, acute erythraemia, erythroleukemia, acute erythraemic myelosis, Di Guglielmo's disease, chronic erythraemia, Heilmeyer-Schöner disease, acute megakaryoblastic leukemia, mast cell leukemia, acute panmyelosis, acute myelofibrosis, and Letterer-Siwe disease.

14. The composition for use according to claim 12, wherein said cancer is a solid cancer which is preferably selected from breast cancer, lung cancer, ovarian cancer, colorectal cancer, colon cancer, pancreatic cancer, bladder cancer, prostate cancer, head and/or neck cancer, and soft-tissue sarcoma.

15. A composition as defined in any one of claims 1 to 11 for use in the treatment of an autoimmune disease/disorder, rheumatoid arthritis, multiple sclerosis, lupus erythematosus, or a neurodegenerative disease/disorder, or for the use in immunomodulatory therapy, wherein the composition is to be administered orally.

16. The composition for use according to any one of claims 12 to 14, wherein the composition is to be administered in combination with a further anticancer agent and/or radiotherapy,
wherein said further anticancer agent is preferably selected from etoposide, fludarabine, mitoxantrone, methotrexate, prednisone, vincristine, and an anti-CD20 monoclonal antibody, and wherein said further anticancer agent is more preferably an anti-CD20 monoclonal antibody selected from rituximab, ocrelizumab, ofatumumab, obinutuzumab, and ibritumomab tiuxetan.

17. The composition for use according to any one of claims 12 to 15, wherein the composition is to be administered in combination with rituximab.

18. The composition for use according to any one of claims 12 to 17, wherein the composition is to be administered in combination with an antiemetic agent,
wherein said antiemetic agent is preferably selected from alosetron, azasetron, bemesetron, cilansetron, clozapine, dazopride, dolasetron, granisetron, lerisetron, metoclopramide, mianserin, mirtazapine, olanzapine, ondansetron, palonosetron, quetiapine, ramosetron, ricasetron, tropisetron, zatosetron, clozapine, cyproheptadine, hydroxyzine, olanzapine, risperidone, ziprasidone, dronabinol, nabilone, tetrahydrocannabinol, alizapride, bromopride, chlorpromazine, clebopride, domperidone, haloperidol, hydroxyzine, itopride, metoclopramide, metopimazine, prochlorperazine, thiethylperazine, trimethobenzamide, cyclizine, dimenhydrinate, diphenhydramine, hydroxyzine, meclizine, promethazine, atropine, diphenhydramine, hyoscyamine, scopolamine, aprepitant, casopitant, ezlopitant, fosaprepitant, maropitant, netupitant, rolapitant, vestipitant, cerium oxalate, dexamethasone, lorazepam, midazolam, propofol, and a combination thereof.

19. The composition for use according to any one of claims 1 or 3 to 17 or the oral pharmaceutical composition of any one of claims 2 to 11, wherein the composition is to be administered orally to a human subject.

20. Use of bendamustine or a pharmaceutically acceptable salt or solvate thereof in combination with a modified cyclodextrin for the preparation of a medicament for oral administration, wherein said modified cyclodextrin is selected from α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

21. A method of treating a disease or disorder in a subject in need thereof, the method comprising orally administering a pharmaceutical composition comprising bendamustine or a pharmaceutically acceptable salt or solvate thereof in combination with a modified cyclodextrin to the subject, wherein said modified cyclodextrin is selected from the group consisting of α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.

22. A method of delivering bendamustine or a pharmaceutically acceptable salt or solvate thereof to a subject in need thereof, the method comprising orally administering a pharmaceutical composition comprising bendamustine or a pharmaceutically acceptable salt or solvate thereof in combination with a modified cyclodextrin to the subject, wherein said modified cyclodextrin is selected from the group consisting of α-cyclodextrin, β-cyclodextrin and γ-cyclodextrin, and wherein said α-cyclodextrin, said β-cyclodextrin or said γ-cyclodextrin is substituted with C₁₋₄ alkyl, hydroxy-C₁₋₄ alkyl, dihydroxy-C₁₋₄ alkyl, —CO(C₁₋₄ alkyl), or any combination thereof.
